(19) United States
(12) Patent Application Publication
(10) Pub. No.: US 2022/0401657 A1
(43) Pub. Date: Dec. 22, 2022

(54) DRUG DELIVERY DEVICE SENSING MODULES

(71) Applicant: JANSSEN BIOTECH, INC., Horsham, PA (US)

(72) Inventors: Peter KRULEVITCH, Pleasanton, CA (US); Anthony R. DIUBALDI, Jackson, NJ (US); Francesco N. ALBERTINI, Pleasanton, CA (US); Sanjay JAIN, Chester Springs, PA (US); Bradley SARGENT, Mission Viejo, CA (US); Jared NATHANSON, Mission Viejo, CA (US); Kevin CHRISTOPHER, Laguna Hills, CA (US)

(21) Appl. No.: 17/776,832

(22) PCT Filed: Nov. 13, 2020
(86) PCT No.: PCT/IB2020/060710

§ 371 Date: May 13, 2022

Related U.S. Application Data
(60) Provisional application No. 62/934,607, filed on Nov. 13, 2019.

(51) Int. Cl. A61M 5/315; A61M 5/20

(52) U.S. Cl. CPC A61M 5/31568; A61M 5/20; A61M 5/31548; A61M 2205/50

(57) ABSTRACT

Various exemplary drug delivery device sensing modules and methods of using drug delivery device sensing modules are provided. In general, a sensing module can be configured to be attached to a drug delivery device configured to deliver a drug. The drug delivery device can be any of a variety of types of drug delivery devices, such as a syringe, an injection device (e.g., an autoinjector, a jet injector, and an infusion pump), a nasal delivery device, and an inhaler. The sensing module can be configured to gather data for one or more parameters related to drug delivery and to transmit data indicative of the gathered data to an external source configured to analyze the data received from the sensing module.

### CROSS REFERENCE TO RELATED APPLICATIONS

The present application claims priority to U.S. Prov. Pat. App. No. 62/934,607 entitled “Drug Delivery Device Sensing Modules” filed Nov. 13, 2019, which is hereby incorporated by reference in its entirety.

### FIELD

The present disclosure relates generally to drug delivery device sensing modules.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

In relation to how a drug is administered to the patient, there are various dosage forms that can be used. For example, these dosage forms may include parenteral, pulmonary, oral, ophthalmic, topical and suppository forms of one or more drugs.

The dosage forms can be administered directly to the patient via a drug administration device. There are a number of different types of drug administration devices commonly available for delivery of the various dosage forms including: syringes, topical dispensers, nasal delivery devices, injection devices (e.g., autoinjectors, jet injectors, and infusion pumps), and inhalers.

It can be desirable to monitor compliance with the guidance that is associated with the drugs that are administered to a patient in various dosage forms. This can provide assurance that correct procedures are being followed and avoid the adoption of incorrect and potentially dangerous approaches. Further, this can also enable optimization of the administration of the drug to the patient.

However, it can be difficult to determine if a drug is properly administered to a patient via a drug administration device and to monitor compliance. The burden for detecting and for reporting proper drug administration is typically on the patient, which may burden the patient with administrative tasks and/or may not be properly or timely reported to a medical professional able to address improper drug administration in a timely manner. Similarly, the burden is typically on the patient for tracking and reporting compliance with the guidance provided to the patient by a physician or healthcare provider. Patients may feel uncomfortable reporting actions that do not comply with the guidance, thus resulting in inaccurate data being reported to and considered by a medical professional, which may adversely affect the patient's overall treatment.

Accordingly, there remains a need for monitoring drug administration.

### SUMMARY

In general, drug delivery device sensing modules and methods of using drug delivery device sensing modules are provided.

In one aspect, a sensing module for a drug delivery device is provided herein. In one embodiment, the sensing module includes a base configured to be attached to an outer surface of a drug delivery device, and a sensor located on the base and configured to gather data regarding at least one of date, time, vibration, temperature, sound, motion, humidity, pressure, fluid level, force, location, proximity, and spatial orientation. The sensing module also includes a communication interface located on the base and configured to wirelessly transmit data to an external source, and a processor located on the base and configured to receive data from the sensor indicative of the gathered data and to cause the communication interface to wirelessly transmit data indicative of the received data to the external source.

The sensing module can vary in any number of ways. For example, the sensing module can also include a flexible circuit board with the sensor, the processor, and the communication interface thereon. For another example, the sensing module can also include a rigid circuit board with the sensor, the processor, and the communication interface thereon.

For yet another example, the base can include a housing with the sensor, the processor, and the communication interface disposed therein. In at least some embodiments, the sensing module can also include a circuit board with the sensor, the processor, and the communication interface thereon, and the circuit board can be disposed within the housing.

For another example, the base can include a thin-film device, and the sensing module can include an adhesive configured to attach the thin-film device to the outer surface of the drug delivery device. For yet another example, the base can be configured to be non-removably attached to the outer surface of the drug delivery device.

For still another example, the sensing module can also include a power source configured to provide power to at least one of the sensor, the processor, and the communication interface. In at least some embodiments, the sensing module can also include an insulator or a conductive trace. The insulator can be attached to the base in a first position, in which the insulator prevents the power source from providing the power to at least one of the sensor, the processor, and the communication interface. The insulator can be configured to be manually moved by a user from the first position to a second position, in which the insulator allows the power source to provide the power to at least one of the sensor, the processor, and the communication interface. The insulator can include a tab configured to be manually torn to move from the first position to the second position, and/or the sensing module can include a switch operatively connected to the power source and with the insulator in the first position the switch can be in an open position and with the insulator in the second position the switch can be in a closed position. The insulator can include a first tab, the sensing module can also include a second tab attached to the base in a third position, in which the sensor is not gathering the data, the second tab can be configured to be manually moved by a user from the third position to a fourth position, and the movement of the second tab from the third position to the fourth position can allow the sensor to begin gathering the data. The conductive trace can be configured to be manually moved by a user from a first position, in which the conductive trace prevents the power source from providing the power to at least one of the sensor, the processor, and the communication interface to a second position, in which the conductive trace allows the power source to provide the power to at least one of the sensor, the processor, and the communication interface. The conductive trace can be on a tab configured to be manually torn to move the conductive trace from the first position to the second position, and/or the sensing module can include a switch operatively connected to the power source and with the conductive trace in the first position the switch can be in an open position and with the conductive trace in the second position the switch can be in a closed position.

For another example, the sensor can include an accelerometer configured to gather data regarding vibration and spatial orientation. For still another example, the sensor can include a temperature sensor configured to gather data regarding temperature. For yet another example, the sensor can be configured to gather data regarding date, time, and at least one of vibration, temperature, sound, motion, humidity, pressure, fluid level, force, location, proximity, and spatial orientation. For another example, the drug delivery device can either be a drug delivery device containing a drug therein configured to be delivered from the drug delivery device or a drug delivery training device configured to simulate drug delivery therefrom.

In another aspect, a drug delivery system is provided that in one embodiment includes a drug delivery device, and a sensing module configured to be attached to an outer surface of the drug delivery device. The sensing module includes a sensor configured to gather data regarding at least one of date, time, vibration, temperature, sound, motion, humidity, pressure, fluid level, force, location, proximity, and spatial orientation, a communication interface configured to wirelessly transmit data to an external source, and a processor configured to receive data from the sensor indicative of the gathered data and to cause the communication interface to wirelessly transmit data indicative of the received data to the external source.

The system can vary in any number of ways. For example, the system can include a flexible circuit board with the sensor, the processor, and the communication interface thereon. For another example, the system can include a rigid circuit board with the sensor, the processor, and the communication interface thereon.

For yet another example, the sensing module can include a housing with the sensor, the processor, and the communication interface disposed therein, and the housing can be attached to the outer surface of the drug delivery device. In at least some embodiments, the sensing module can include a circuit board with the sensor, the processor, and the communication interface thereon, and the circuit board can be disposed within the housing.

For still another example, the sensing module can include a thin-film device, and the system can include an adhesive configured to attach the thin-film device to the outer surface of the drug delivery device. For another example, the sensing module can be non-removably attached to the outer surface of the drug delivery device.

For yet another example, the sensing module can include a power source configured to provide power to at least one of the sensor, the processor, and the communication interface. In at least some embodiments, the system can also include an insulator or a conductive trace. The insulator can be in a first position, in which the insulator prevents the power source from providing the power to at least one of the sensor, the processor, and the communication interface, and the insulator can be configured to be manually moved by a user from the first position to a second position, in which the insulator allows the power source to provide the power to at least one of the sensor, the processor, and the communication interface. The drug delivery device can include a cap configured to be manually removed by a user from a housing of the drug delivery device, and the removal of the cap can be configured to cause the insulator to automatically move from the first position to the second position. The drug delivery device can include a trigger configured to be manually actuated by a user to trigger drug delivery from the drug delivery device, and the actuation of the trigger can be configured to cause the insulator to automatically move from the first position to the second position. The insulator can include a tab configured to be manually torn to move from the first position to the second position. The system can include a switch operatively connected to the power source, and with the insulator in the first position the switch can be in an open position and with the insulator in the second position the switch can be in a closed position. The insulator can include a first tab, the system can also include a second tab attached to the base in a third position, in which the sensor is not gathering the data, the second tab can be configured to be manually moved by a user from the third position to a fourth position, and the movement of the second tab from the third position to the fourth position can allow the sensor to begin gathering the data. The conductive trace can be configured to be manually moved by a user from a first position, in which the conductive trace prevents the power source from providing the power to at least one of the sensor, the processor, and the communication interface to a second position, in which the conductive trace allows the power source to provide the power to at least one of the sensor, the processor, and the communication interface. In at least some embodiments, the drug delivery device can include a cap configured to be manually removed by a user from a housing of the drug delivery device, and the removal of the cap can be configured to cause the power source to begin providing power to the at least one of the sensor, the processor, and the communication interface. The drug delivery device can include a cap configured to be manually removed by a user from a housing of the drug delivery device, and the removal of the cap can be configured to cause the conductive trace to automatically move from the first position to the second position. The drug delivery device can include a trigger configured to be manually actuated by a user to trigger drug delivery from the drug delivery device, and the actuation of the trigger can be configured to cause the conductive trace to automatically move from the first position to the second position. The conductive trace can be on a tab configured to be manually torn to move from the first position to the second position. The system can include a switch operatively connected to the power source, and with the conductive trace in the first position the switch can be in an open position and with the insulator in the second position the switch can be in a closed position.

For still another example, the sensor can include an accelerometer configured to gather data regarding vibration and spatial orientation. For another example, the sensor can include a temperature sensor configured to gather data regarding temperature. For yet another example, the sensor can be configured to gather data regarding date, time, and at least one of vibration, temperature, sound, motion, humidity, pressure, fluid level, force, location, proximity, and spatial orientation. For still another example, the drug delivery device can either be a drug delivery device containing a drug therein configured to be delivered from the drug delivery device or a drug delivery training device configured to simulate drug delivery therefrom. For another example, the drug can include one of infliximab, golimumab, ustekinumab, daratumumab, guselkumab, epoetin alfa, risperidone, and paliperidone palmitate.

In another aspect, a method of using a drug delivery device is provided that in one embodiment includes gathering, using a sensor of a sensing module attached to an outer surface of a drug delivery device configured to deliver a drug, data regarding at least one of date, time, vibration, temperature, sound, motion, humidity, pressure, fluid level, force, location, proximity, and spatial orientation. The method also includes causing, using a processor of the sensing module, a communication interface of the sensing module to wirelessly transmit data indicative of the gathered data to a source external to the drug delivery device and external to the sensing module.

The method can vary in any number of ways. For example, a power source of the sensing module can begin providing power to at least one of the sensor and the processor in response to a cap of the drug delivery device being manually removed by a user from a housing of the drug delivery device. In at least some embodiments, the removal of the cap can cause an insulator to be removed from an electrical path between the power source and the at least one of the sensor and the processor, or the removal of the cap can cause a conductive trace between the cap and the sensing module to become disconnected. In at least some embodiments, the sensor can begin the gathering of the data in response to the power source beginning to provide power to the at least one of the sensor and the processor. In at least some embodiments, the sensor can begin the gathering of the data in response to a tab being manually removed by a user from the drug delivery device.

For another example, a power source of the sensing module can begin providing power to at least one of the sensor and the processor in response to a trigger of the drug delivery device being manually actuated by a user. In at least some embodiments, the actuation of the trigger can cause an insulator to be removed from an electrical path between the power source and the at least one of the sensor and the processor, or the actuation of the trigger can cause a conductive trace to become disconnected. In at least some embodiments, the sensor can begin the gathering of the data in response to the power source beginning to provide power to the at least one of the sensor and the processor. In at least some embodiments, the sensor can begin the gathering of the data in response to a tab being manually removed by a user from the drug delivery device.

For another example, the sensor can include an accelerometer that gathers data regarding vibration and spatial orientation. For yet another example, the sensor can include a temperature sensor that gathers data regarding temperature. For still another example, the sensor can gather data regarding date, time, and at least one of vibration, temperature, sound, motion, humidity, pressure, fluid level, force, location, proximity, and spatial orientation. For another example, the data can be gathered during delivery of the drug from the drug delivery device. For yet another example, the data can be gathered prior to starting delivery of the drug from the drug delivery device.

For another example, the method can include causing a computer system that is external to the drug delivery device to provide instructions for using the drug delivery device during delivery of the drug from the drug delivery device, and the instructions can be based on data gathered using the sensor. The instructions can be provided via an app.

For still another example, the drug can include one of infliximab, golimumab, ustekinumab, daratumumab, guselkumab, epoetin alfa, risperidone, and paliperidone palmitate.

In another aspect, a method of using a drug delivery training device is provided that includes gathering, using a sensor of a sensing module attached to an outer surface of a drug delivery training device that simulates delivery of a drug, data regarding at least one of date, time, vibration, temperature, sound, motion, humidity, pressure, fluid level, force, location, proximity, and spatial orientation. The method also includes causing, using a processor of the sensing module, a communication interface of the sensing module to wirelessly transmit data indicative of the gathered data to a source external to the drug delivery training device and external to the sensing module.

The method can have any number of variations. For example, a power source of the sensing module can begin providing power to at least one of the sensor and the processor in response to a cap of the drug delivery training device being manually removed by a user from a housing of the drug delivery training device. In at least some embodiments, the removal of the cap can cause an insulator coupled to the sensing module to be removed from an electrical path between the power source and the at least one of the sensor and the processor, or the removal of the cap can cause a conductive trace between the cap and the sensing module to become disconnected. In at least some embodiments, the sensor can begin the gathering of the data in response to the power source beginning to provide power to the at least one of the sensor and the processor. In at least some embodiments, the sensor can begin the gathering of the data in response to a tab being manually removed by a user from the drug delivery training device.

For another example, a power source of the sensing module can begin providing power to at least one of the sensor and the processor in response to a trigger of the drug delivery training device being manually actuated by a user. In at least some embodiments, the actuation of the trigger can cause an insulator to be removed from an electrical path between the power source and the at least one of the sensor and the processor, or the actuation of the trigger can cause a conductive trace to become disconnected. In at least some embodiments, the sensor can begin the gathering of the data in response to the power source beginning to provide power to the at least one of the sensor and the processor. In at least some embodiments, the sensor can begin the gathering of the data in response to a tab being manually removed by a user from the drug delivery training device.

For another example, the sensor can include an accelerometer that gathers data regarding vibration and spatial orientation. For yet another example, the sensor can include a temperature sensor that gathers data regarding temperature. For still another example, the sensor can gather data regarding date, time, and at least one of vibration, temperature, sound, motion, humidity, pressure, fluid level, force, location, proximity, and spatial orientation. For another example, the drug delivery training device simulates an autoinjector.

For still another example, the method can include causing a computer system that is external to the drug delivery training device to provide instructions for using the drug delivery training device during use of the drug delivery training device, and the instructions can be based on data gathered using the sensor. The instructions can be provided via an app.

### BRIEF DESCRIPTION OF DRAWINGS

The present invention is described by way of reference to the accompanying figures which are as follows:

FIG. 1 is a perspective view of one embodiment of a drug delivery device with one embodiment of a sensing module attached thereto;

FIG. 2 is a perspective view of the sensing module of FIG. 1;

FIG. 3 is a schematic view of the sensing module of FIG. 1;

FIG. 4 is a schematic view of one embodiment of a communication network system;

FIG. 5 is a perspective view of another embodiment of a drug delivery device with another embodiment of a sensing module attached thereto;

FIG. 6 is a bottom view of the sensing module of FIG. 5;

FIG. 7 is a bottom view of a printed circuit board of the sensing module of FIG. 6;

FIG. 8 is a perspective view of the printed circuit board of the sensing module of FIG. 6;

FIG. 9 is a side view of another embodiment of a drug delivery device with the sensing module of FIG. 6 attached thereto;

FIG. 10 is a perspective view of another embodiment of a drug delivery device with one embodiment of a tab and another embodiment of a sensing module attached thereto and with internal components thereof removed for clarity of illustration;

FIG. 11 is another perspective view of the drug delivery device of FIG. 10;

FIG. 12 is a cross-sectional view of the drug delivery device of FIG. 10;

FIG. 13 is an exploded view of the tab and the sensing module of FIG. 10;

FIG. 14 is a perspective view of a printed circuit board of the sensing module of FIG. 13;

FIG. 15 is a perspective view of another embodiment of a tab and another embodiment of a sensing module coupled to the tab;

FIG. 16 is a bottom view of the sensing module and tab of FIG. 15;

FIG. 17 is a perspective view of another embodiment of a drug delivery device with the tab and the sensing module of FIG. 15;

FIG. 18 is another perspective view of the drug delivery device of FIG. 17;

FIG. 18A is a cross-sectional view of one end portion of the drug delivery device of FIG. 17;

FIG. 19 is a side, partial view of the sensing module of FIG. 15;

FIG. 20 is a perspective view of the tab of FIG. 15, a connector, and a partial portion of the sensing module of FIG. 15;

FIG. 21 is a perspective view of the tab of FIG. 15;

FIG. 22 is a perspective view of the connector of FIG. 20;

FIG. 23 is a side, partial view of another embodiment of a sensing module;

FIG. 24 is a perspective view of yet another embodiment of a drug delivery device and yet another embodiment of a sensing module;

FIG. 25 is another perspective view of the drug delivery device of FIG. 24;

FIG. 26 is a perspective view of still another embodiment of a drug delivery device and yet another embodiment of a sensing module;

FIG. 27 is another perspective view of the drug delivery device of FIG. 26;

FIG. 28 is a perspective view of another embodiment of a drug delivery device with another embodiment of a tab and another embodiment of a sensing module attached thereto;

FIG. 29 is another perspective view of the drug delivery device of FIG. 28;

FIG. 30 is a perspective view of the tab and the sensing module of FIG. 28;

FIG. 31 is a perspective view of the tab of FIG. 30;

FIG. 32 is a cross-sectional, partial view of the sensing module of FIG. 30;

FIG. 33 is a perspective view of one end portion of the drug delivery device of FIG. 28;

FIG. 34 is a perspective view of the drug delivery device of FIG. 28 with an outer boot and end cap removed;

FIG. 35 is a perspective view of the drug delivery device of FIG. 28 with the outer boot removed;

FIG. 36 is a perspective, partial view of yet another embodiment of a drug delivery device with yet another embodiment of a tab attached thereto, the tab being in a first position;

FIG. 37 is another perspective, partial view of the drug delivery device of FIG. 36 with the tab in a second position;

FIG. 38 is a perspective view of another embodiment of a drug delivery device with another embodiment of a tab and another embodiment of a sensing module attached thereto;

FIG. 39 is a top view of a printed circuit board of the sensing module of FIG. 38;

FIG. 40 is a top view of a power source of the sensing module of FIG. 38;

FIG. 41 is a bottom view of the tab of FIG. 38;

FIG. 42 is a dual side and front view of one embodiment of a pill bottle with another embodiment of a tab and another embodiment of a sensing module attached thereto;

FIG. 43 is a front view of the pill bottle of FIG. 42 with another embodiment of a tab and another embodiment of a sensing module attached thereto;

FIG. 44 is a view of one embodiment of a drug delivery training app page on a mobile phone;

FIG. 45 is a view of another embodiment of a drug delivery training app page on the mobile phone of FIG. 44;

FIG. 46 is a view of yet another embodiment of a drug delivery training app page on the mobile phone of FIG. 44; and

FIG. 47 is a flowchart of one embodiment of a method of a sensing module establishing communication with an external source.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary drug delivery device sensing modules and methods of using drug delivery device sensing modules are provided. In general, a sensing module can be configured to be attached to a drug delivery device configured to deliver a drug. The drug delivery device can be any of a variety of types of drug delivery devices, such as a syringe, an injection device (e.g., an autoinjector, a jet injector, and an infusion pump), a nasal delivery device, and an inhaler. The sensing module can be configured to gather data for one or more parameters related to drug delivery and to transmit data indicative of the gathered data to an external source configured to analyze the data received from the sensing module. The sensing module may help improve compliance by allowing errors in drug delivery to be identified based on the gathered data and thus provide opportunity for the errors to be addressed and/or by allowing any missed and off-schedule doses for a user to be identified based on the gathered data and thus provide a user's health care provider with information to discuss with the user and/or better analyze the user's treatment. The sensing module may similarly help increase clarity into clinical trial data by allowing errors in drug delivery during the clinical trial to be identified based on the gathered data and thus provide opportunity for the errors to be addressed before completion of the clinical trial and/or by allowing any missed and off-schedule doses for a clinical trial participant to be identified based on the gathered data and thus provide a clinical trial administrator with information to discuss with the clinical trial participant and/or better analyze clinical trial results.

Examples of the parameters related to drug delivery that can be sensed by the sensing module include date, time, vibration, temperature, sound, motion, humidity, pressure, fluid level, force, location, proximity, and spatial orientation. Gathering date data and/or time data may allow for other sensed parameter(s) to be accurately date and/or time stamped, e.g., correlated with a particular date and/or time. Gathering vibration data may allow for detecting when drug delivery has begun, e.g., by detected vibration being indicative of spring or other mechanical action such as advancement of a needle or retraction (manually or automatically caused) of a needle cover sleeve to expose a needle, etc., for detecting when drug delivery has been completed, e.g., by detected vibration being indicative of spring or other mechanical action such as retraction of a needle or advancement (manually or automatically caused) of a needle cover sleeve that locks in place over a needle, etc., for detecting when reconstitution or mixing of the drug to be delivered has begun in the drug delivery device, e.g., by detected vibration being indicative of the drug delivery device being shaken manually by a user to cause the reconstitution or mixing, etc., for detecting when reconstitution or mixing of the drug to be delivered has stopped in the drug delivery device, e.g., by the ceasing of detected vibration being indicative of a stop of the drug delivery device's manual shaking, etc., and/or for evaluating quality of reconstitution or mixing of the drug to be delivered, e.g., by detected vibration being indicative of a shaking force that either meets or fails predetermined shaking force criteria for proper reconstitution or mixing, etc. Gathering temperature data may provide ambient temperature information to indicate whether the drug is at a safe temperature for storage and/or for delivery to a user. Gathering sound data may allow for detecting when drug delivery has begun, e.g., by detected acoustic data within a particular frequency range being indicative of when drug delivery has begun, by detected sound being indicative of an inhaler's drug canister being pressed down, by detected sound being indicative of a needle being advanced through spring or other mechanical action, by a detected click sound being indicative of a trigger of the drug delivery device being pressed, etc., for detecting when drug delivery has been completed, e.g., by detected sound being indicative of spring or other mechanical action retracting a needle, by detected sound being indicative of when aerosol delivery of the drug has stopped, by a detected click sound being indicative of a trigger of the drug delivery device being released, etc., for detecting when reconstitution or mixing of the drug to be delivered has begun in the drug delivery device, e.g., by detected sound being indicative of activation of the device's reconstitution or mixing mechanism, etc., and/or for detecting when reconstitution or mixing of the drug to be delivered has stopped in the drug delivery device, e.g., by the ceasing of detected sound being indicative of deactivation of the device's reconstitution or mixing mechanism, etc. Gathering motion data may allow for detecting when drug delivery has begun, e.g., by detected motion being indicative of an inhaler's drug canister being pressed down, of a plunger being pushed down, etc., and/or for detecting when drug delivery has been completed, e.g., by detected motion being indicative of an inhaler's drug canister being released to allow the canister to move up to a resting position, the drug delivery device being lifted manually from an injection site, etc. Gathering humidity data may provide information as to whether the drug is at a safe humidity level for storage and/or for delivery to a user. Gathering pressure data may allow for detecting when drug delivery has begun, e.g., by detected pressure being indicative of spring or other mechanical action advancing a needle, of an inhaler's drug canister being pressed down, of a plunger being pushed down, etc., and/or for detecting when drug delivery has been completed, e.g., by detected pressure being indicative of spring or other mechanical action retracting a needle, of an inhaler's drug canister being released to allow the canister to move up to a resting position, etc. Gathering fluid level data may allow for detecting the presence of liquid drug in the drug delivery device's reservoir, which may be indicative of no drug delivery having yet occurred from the device, and/or for detecting the absence of liquid drug in the drug delivery device's reservoir, which may be indicative of drug delivery having occurred. Gathering force data may allow for detecting a force with which the drug delivery device as an injection device is being held against a patient (e.g., against the patient's skin), which may help inform whether drug injection fails since too low a force can cause injection failure. Gathering location data may allow for detecting a geographic location of a patient, which may allow for other sensed parameter(s) to be accurately location stamped, e.g., correlated with a particular location. Gathering proximity data may allow for detecting a distance of the drug delivery device from skin before, during, and/or after delivery of the drug from the drug delivery device, which may help indicate whether the drug delivery device is being held against skin while the drug is being delivered, e.g., as with an injection device intended to be held against a skin surface during drug delivery, and/or whether the drug delivery device is removed from skin before delivery of the drug has completed, e.g., as with an injection device intended to be removed from a skin surface after delivery of the drug has completed. Gathering spatial orientation data may allow for detecting the drug delivery device's orientation relative to ground when the drug is delivered, which may be indicative of whether the drug was properly administered, and/or for evaluating quality of reconstitution or mixing of the drug to be delivered, e.g., by detected spatial orientations over a period of time being indicative of a number of inversions of the drug delivery device that either meets or fails predetermined inversion number criteria for proper reconstitution or mixing, etc.

Further discussion of gathering data and using gathered data in determining proper reconstitution or mixing for drug delivery devices and drug delivery training devices are provided in U.S. Pat. Pub. No. 2018/0182263 entitled “Devices And Methods For Drug Administration And Mixing, And Training Of Proper Techniques Therefor” published Jun. 28, 2018, U.S. Pat. Pub. No. 2018/0190153 entitled “Devices And Methods For Drug Administration And Mixing, And Training Of Proper Techniques Therefor” published Jul. 5, 2018, U.S. Pat. Pub. No. 2018/0190154 entitled “Devices And Methods For Drug Administration And Mixing, And Training Of Proper Techniques Therefor” published Jul. 5, 2018, and U.S. Pat. Pub. No. 2019/00433386 entitled “Devices And Methods For Drug Administration And Mixing, And Training Of Proper Techniques Therefor” published Feb. 7, 2019, which are hereby incorporated by reference in their entireties.

In an exemplary embodiment, the sensing module is configured to be attached to an outer surface of the drug delivery device. The sensing module being attachable to a drug delivery device's outer surface may safely isolate the sensing module's electronic components from the drug contained in the drug delivery device and from the drug delivery device's drug delivery components (e.g., needle, syringe, plunger, pump, pressurized drug canister, etc.). The sensing module being attachable to a drug delivery device's outer surface may facilitate use of the sensing module with existing drug delivery devices because the drug delivery device need not be modified in order to accommodate the sensing module. The sensing module may simply be attached to an outer surface of the drug delivery device and thus may allow for drug delivery devices to be designed without needing to reserve valuable, limited internal real estate within the drug delivery device for the sensing module since the sensing module may simply be attached to an outer surface of the drug delivery device. The sensing module being attachable to a drug delivery device's outer surface may ease incorporation of the sensing module into a drug delivery device's manufacturing process since the sensing module can be attached to the drug delivery device's outer surface after the drug delivery device has otherwise been assembled.

The drug to be delivered using the drug delivery device having the sensing module thereto can be any of a variety of drugs. Examples of drugs that can be delivered using a drug delivery device as described herein (or trained for delivery using a drug delivery training device as described herein) include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), and Invega Trinza® (paliperidone palmitate).

The sensing module can be configured to be attached to a drug delivery training device configured to simulate delivery of a drug for training purposes. The sensing module may facilitate the training of users to properly use a drug delivery device since the sensing module's use with a drug delivery training device may provide insight into various factors affecting proper drug delivery, including whether a user is using the drug delivery device correctly and whether the user is adhering to the intended drug delivery schedule. The drug delivery training device to which the sensing module can be attached can be any of a variety of types of drug delivery training devices, such as a syringe, an injection device (e.g., an autoinjector, a jet injector, and an infusion pump), a nasal delivery device, and an inhaler. The sensing module used with a drug delivery training device is configured and used similar to that discussed herein for a drug delivery device configured to deliver a drug.

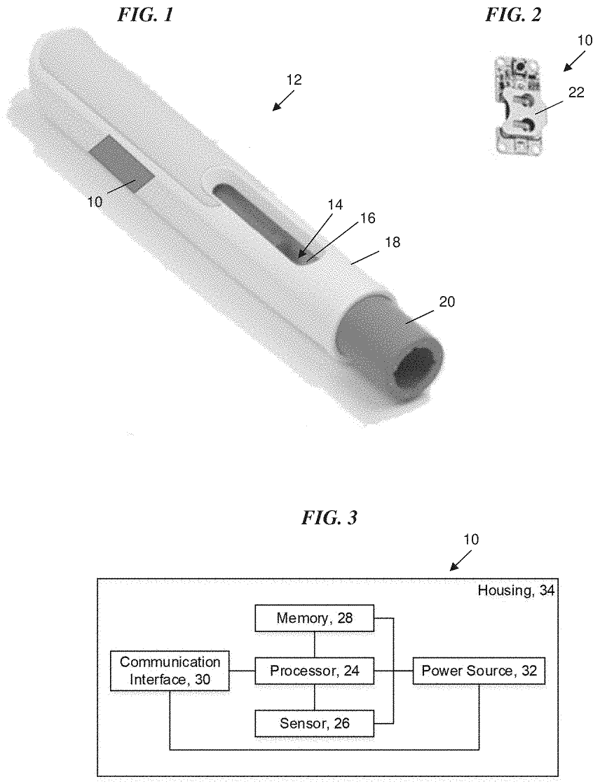

FIGS. 1 and 2 illustrate one embodiment of a sensing module 10 configured to gather data for one or more parameters related to drug delivery and to transmit data indicative of the gathered data to an external source configured to analyze the data received from the sensing module 10. FIG. 2 illustrates the sensing module 10 as a standalone element. FIG. 1 illustrates the sensing module 10 attached to one embodiment of a drug delivery device 12 configured to deliver a drug 14, which is a clear liquid in this illustrated embodiment. The drug delivery device 12 in this illustrated embodiment is an autoinjector configured to inject the drug 14 from a container 16 in a housing 18 of the device 12 and out of a needle (obscured in FIG. 1 by a needle shield 20 of the device 12).

The sensing module 10 is attached to an outer surface of the device 12. The outer surface is an outer surface of the housing 18, but the sensing module 10 can be attachable to another outer surface of a drug delivery device, such as on a depressible head of the device, on a rotatable dose setter of the device, a trigger of the device, etc. The sensing module 10 is attached to the device 12 with an adhesive 22, e.g., a layer of an adhesive on the sensing module 10, in this illustrated embodiment. For example, the sensing module 10 can be a thin label-type device similar to a sticker in which the adhesive 22 is on one side of the thin label that is attached to the device 12 or is on both sides of the thin label (similar to double-sided tape) with one side adhered to the sensing module 10 and one side adhered to the device 12. For another example, the sensing module 10 can include a small box or other small housing with the adhesive 22 on one side thereof. The sensing module 10 being attachable to a drug delivery device using adhesive (with a thin label-type device or a small housing) facilitates retrofitting existing drug delivery devices with the sensing module 10 since the existing drug delivery device need not be modified in any way to accommodate the attachment of the sensing module 10 thereto. However, the sensing module 10 can be attached to a drug delivery device in other ways, such as by being press fit into a cavity formed in an outer surface of a drug delivery device (e.g., by the sensing module 10 including a small box or other small housing having a size and shape configured to be securely press fit into the cavity), by including one or more protrusions configured to snap or otherwise fit into one or more corresponding holes formed in an outer surface of a drug delivery device (e.g., by the sensing module 10 including a small box or other small housing that includes the one or more protrusions), or by including one or more holes configured to receive therein one or more corresponding protrusions extending from an outer surface of a drug delivery device (e.g., by the sensing module 10 including a small box or other small housing that includes the one or more protrusions). In some embodiments, more than one type of attachment mechanism can be used to attach a sensing module to a drug delivery device to provide redundancy to help ensure that the sensing module remains attached to the drug delivery device through final use of the drug delivery device. For example, a sensing module can include an adhesive layer and an additional attachment mechanism (e.g., one or more protrusions, one or more holes, a body configured to be press fit into a cavity of the drug delivery device, etc.). For another example, a sensing module can include one or more protrusions and one or more holes.

In an exemplary embodiment, as in the illustrated embodiment of FIG. 1, the sensing module 10 is non-removably attached to the drug delivery device 12, which may help ensure that the sensing module 10 is always available to gather data, that the sensing module 10 is not reused, and/or that the sensing module 10 is attached properly to the device 12 by being attached thereto as part of a manufacturing process before the device 12 is shipped for provision to a user. In embodiments (discussed below) in which a sensing module includes a tamper-resistant feature, the sensing module being non-removably attached to a drug delivery device may help ensure that the sensing module accurately provides evidence of tampering or no tampering. In other embodiments a sensing module can be removably attached to a drug delivery device, which may facilitate use of the sensing module with multiple drug delivery devices, such as with each of a plurality of single-dose drug delivery devices for the same user or with each successive multi-dose drug delivery device used by the same user. A sensing module configured to be removably attached to a drug delivery device can be provided to a user already adhered to the drug delivery device, or the sensing module can be configured to be adhered to the drug delivery device by a user, in which case the sensing module can include a removable protective layer of paper, plastic, etc. configured to be removed by a user to expose adhesive for attachment of the sensing module to the drug delivery device.

The sensing module 10 includes a variety of electronic components to facilitate the gathering of data and the transmission of gathered data to an external source. FIG. 3 illustrates one embodiment of the sensing module's electronic components. The sensing module 10 includes a processor 24, a sensor 26 configured to gather data regarding one or more parameters and transmit the gathered data to the processor 24, a memory 28 configured to receive data from the processor 24 for storage in the memory 28 and configured to store instructions therein that are executable by the processor 24, a communication interface 30 configured to transmit data to an external source at the instruction of the processor 24, and a power source 32 configured to provide power to one or more of the sensing module's other electronic components.

In an exemplary embodiment, the sensing module's electronic components are mechanically supported on a printed circuit board (PCB) and electrically connected to one another as needed on the PCB. To facilitate the electrical connections, the PCB can include a bus system, e.g., one or more separate physical buses, communication lines/interfaces, and/or multi-drop or point-to-point connections, connected by appropriate bridges, adapters, and/or controllers. The PCB can be flexible, which may facilitate attachment of the sensing module 10 to a curved surface of a drug delivery device. Alternatively, the PCB can be rigid, which may provide durability to the sensing module 10. Whether rigid or flexible, the PCB can be disposed in a housing 34. The housing 34 can define a base of the sensing module 10 configured to be attached to the outer surface of the drug delivery device 12 using one or more attachment mechanisms as described herein. The housing 34 containing the sensing module's electronic components therein can help protect the electronic components from damage.

The processor 24 can include any type of microprocessor or central processing unit (CPU), including programmable general-purpose or special-purpose microprocessors and/or any one of a variety of proprietary or commercially available single or multi-processor systems. In an exemplary embodiment the processor 24 is a single processor, which may help control cost and/or size of the sensing module 10.

The memory 28 is configured to provide storage for data, e.g., instructions (e.g., code) to be executed by the processor 24 and data gathered by the sensor 26. The memory 28 can include storage using, e.g., read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The communication interface 30 (also referred to herein as a “network interface”) is configured to enable communication over a network with sources external to the sensing module 10 and the drug delivery device 12 to which the sensing module 10 is attached. In an exemplary embodiment the communication interface 30 is configured to communicate wirelessly using any of a number of wireless techniques, e.g., Wi-Fi, Near Field communication (NFC), Bluetooth, Bluetooth Low Energy (BLE), cellular communication, etc. In one exemplary embodiment, the communication interface 30 is configured to communicate wirelessly using BLE. In another exemplary embodiment, the communication interface 30 is configured to communicate wirelessly using Bluetooth. In yet another exemplary embodiment, the communication interface 30 is configured to communicate wirelessly using NFC. In still another exemplary embodiment, the communication interface 30 is configured to communicate wirelessly using each of NFC and BLE. In still another exemplary embodiment, the communication interface 30 is configured to communicate wirelessly using each of NFC and Bluetooth.

The communication interface 30 being configured to communicate wirelessly using NFC, as the communication interface's only wireless capability or as one of a plurality of wireless capabilities of the communication interface 30 (e.g., NFC and BLE, NFC and Bluetooth, etc.), may allow for data stored at the sensing module 10, e.g., in the memory 28, to be retrieved from the sensing module 10 even if the power source 32 has been deleted of power or lacks sufficient power to allow for communication from the communication interface 30, for example if the power source 32 as a battery has run out of battery power or lacks sufficient battery power to allow for communication from the communication interface 30. NFC technology allows a data source to wirelessly receive energy from a data destination. Accordingly, the communication interface 30 being configured to communicate using NFC allows the communication interface 30 to receive power from the external source, e.g., from an NFC reader, such that data stored at the sensing module 10 can be communicated from the communication interface 30 using NFC even if the power source 32 has been deleted of power or lacks sufficient power to allow for communication from the communication interface 30.

The power source 32 may run out of power or have an insufficient power supply for communication from the communication interface 30 before all desired data has been retrieved from the sensing module 10 for any of a variety of reasons. For example, the communication interface 30 may be out of range of the external source until after the power source 32 has been depleted of power. For another example, the sensing module 10 including the power source 32 may have been manufactured long enough ago that the power source 32 was depleted of power before all desired data could be retrieved from the sensing module 10. For yet another example, the power source 32 may have become damaged and/or otherwise experienced an error preventing the power source 32 from providing power as needed for data to be communicated from the sensing module 10 to the external source.

The communication interface 30 being configured to communicate wirelessly using NFC, as the communication interface's only wireless capability or as one of a plurality of wireless capabilities of the communication interface 30 (e.g., NFC and BLE, NFC and Bluetooth, etc.), may allow for data to be stored on the sensing module 10, e.g., in the memory 28, as part of the sensing module's manufacturing process and/or at other time(s) before a user begins using a drug delivery device to which the sensing module 10 is attached. NFC technology allows data to be communicated from the external source, e.g., an NFC reader, to the communication interface 30 for storage on the sensing module 10. For example, drug study or clinical trial data can be stored on the sensing module 10 related to a drug study or clinical trial in which a drug delivery device having the sensing module 10 attached thereto will be used. Drug study or clinical trial data can thus be retrieved from the sensing module 10 to, e.g., help ensure that the sensing module's data is correctly associated with the drug study or clinical trial and/or to help verify that the drug and/or drug delivery device complies with requirements of the drug study or clinical trial. Examples of drug study or clinical trial data include drug type or name, drug expiration date, drug manufacture date, drug study or clinical trial number, etc. For another example, drug delivery device data can be stored on the sensing module 10. Drug delivery device data can thus be retrieved from the sensing module 10 to identify the drug delivery device to which the sensing module 10 is attached, which may facilitate compliance analysis and/or analysis of correct device usage. Examples of drug delivery device data include drug delivery device type or name, drug delivery data manufacture date, manufacturing site, device identification number or code, etc.

The sensing module 10 can include any of a variety of other software and/or hardware components not shown in FIG. 3. For example, the sensing module 10 can include an LED or other light to show sensing status of the sensing module 10 (e.g., light on when the sensor 26 is gathering data and light off when the sensor 26 is not gathering data, etc.), to show power status of the sensing module 10 (e.g., light on when the power source 32 is providing power to one or more components of the sensing module 10 and light off when the power source 32 is not providing power to one or more components of the sensing module 10, etc.), and/or to show other information (e.g., a light in one color before drug delivery begins and in a different color after drug delivery, etc.). For another example, the sensing module 10 can include a speaker configured to provide audio to a user (e.g., a beep when the sensing module 10 is powered on, a beep when drug delivery begins, a beep when drug delivery ends, a beep indicating low power, etc.). For yet another example, the sensing module 10 can include a graphic and/or text display configured to provide graphic and/or text information to a user (e.g., graphic and/or text indicating that the sensing module 10 has been powered on, graphic and/or text indicating that drug delivery has started, graphic and/or text indicating that drug delivery is in progress, graphic and/or text indicating that drug delivery has ended, graphic and/or text indicating low power, etc.). The sensing module 10 including a user interface that includes a light, a speaker, and/or a graphic and/or text display may allow a user to receive information that may otherwise be provided to the user via an app on a mobile phone (or other computer system), such as when the user does not have access to the app at all or temporarily lacks access to the app.

In other embodiments, the sensing module may differ in architecture and operation from that shown and described in FIG. 3. For example, the sensor 26 and communication interface 30 can be integrated together. For another example, the processor 24 and communication interface 30 can be integrated together. For yet another example, the sensor 26 can include its own local memory in addition to the sensing module 10 including the memory 28. For still another example, the power source can be off-board the sensing module 10. For another example, the sensor 26, communication interface 30, and processor 24 can be integrated together. For still another example, the housing 34 can include multiple housings that each house therein at least one component of the sensing module 10, e.g., a first housing that houses the communication interface 30 and a second housing that houses the remaining sensing module components, a first housing that houses the sensor 26 and a second housing that houses the remaining sensing module components, a first housing that houses the sensor 26 and the communication interface 30 and a second housing that houses the remaining sensing module components, a first housing that houses the power source 32 and a second housing that houses the remaining sensing module components, etc. Using multiple housings allows the housings to be attached to the drug delivery device at different locations and may allow for each of the housings to be smaller than if a single housing was used and thereby facilitate attachment of the housings to smaller parts of the drug delivery device and/or make the sensing module 10 less likely to interfere with a user's handling of the drug delivery device.

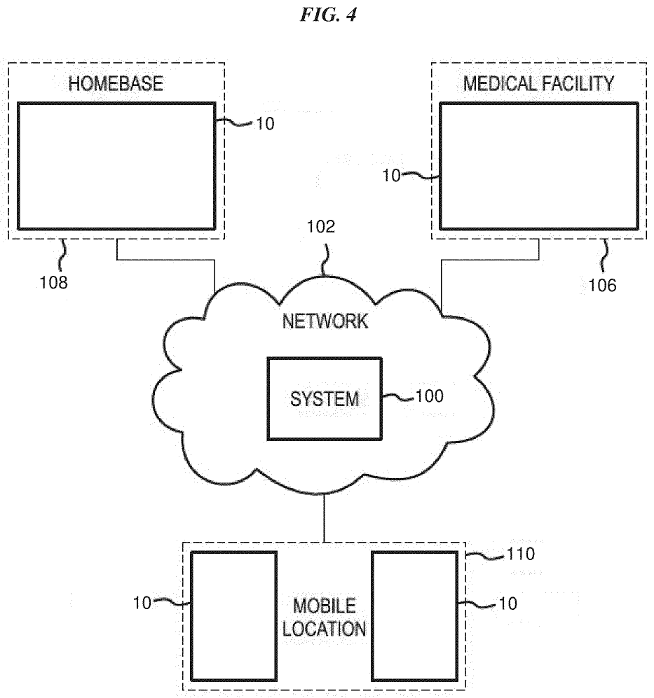

The communication interface 30 is configured to communicate with an external source such as a computer system located remotely from the sensing module 10, such as a central computer system 100 shown in FIG. 4. As shown in FIG. 4, the communication interface 30 is configured to communicate with the central computer system 100 through a communication network 102 from any number of locations where the sensing module 10 attached to the drug delivery device 12 may be located, such as a medical facility 106, e.g., a hospital or other medical care center, a home base 108 (e.g., a patient's home or office or a care taker's home or office), or a mobile location 110. In some embodiments, the central computer system 100 can be located at a same location as the communication interface 30 but be remotely located from the central computer system at that location, e.g., the communication interface 30 being in one room of the home base 108 or medical facility 106 and the central computer system 100 being in another room of the home base 108 or medical facility 106.

The communication interface 30 can be configured to access the system 100 through a wired and/or wireless connection to the network 102. In an exemplary embodiment the communication interface 30 is configured to access the system 100 wirelessly using any of a number of wireless techniques, which can facilitate accessibility of the system 100 from almost any location in the world where the sensing module 10 attached to the drug delivery device 12 may be located. A person skilled in the art will appreciate that communications over the network 102 can include security features to help protect unauthorized access to transmitted data and/or to nodes within the network 102.

The central computer system 100 can have any of a variety of configurations, as will be appreciated by a person skilled in the art, including components such as a processor, a communication interface, a memory, an input/output interface, and a bus system. The computer system 100 can also include any of a variety of other software and/or hardware components, including by way of non-limiting example, operating systems and database management systems. The central computer system 100 can be any of a variety of types of computer systems, such as a desktop computer, a workstation, a minicomputer, a laptop computer, a tablet computer, a personal digital assistant (PDA), a mobile phone, a smart watch, etc.

The computer system 100 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 100 can also include a web server for generating and/or delivering the web pages to client computer systems. The presented pages and/or streams may allow a user of the computer system 100 to view data received from the sensing module 10 and/or analysis of the data as performed by the computer system 100.

In general, the sensor 26 is configured to gather data regarding at least one parameter and to transmit the data to the processor 24. The processor 24 is configured to cause the data received from the sensor 26 to be transmitted to the communication interface 30, either from the processor 24 before (or without) storage of the data in the memory 28 or from the memory 28 after causing the data to be stored in the memory 28. The data may not be stored in the memory 28 if, e.g., the memory 28 has limited storage space. The communication interface 30 is configured to transmit the data to the external source, e.g., the central computer system 100, for review by a user and/or for analysis by a processor of the central computer system 100 for review by a user. In some embodiments, the processor 24 can be configured to analyze the data instead of or in addition to a processor of the central computer system 100 analyzing the data. The central computer system 100 may be more robust than the computer system of the drug delivery device 12, and thus the processor of the central computer system 100 may have more processing power than the processor 24 of the drug delivery device 12 and/or be more capable of analyzing large amounts of data.

In an exemplary embodiment, data is transmitted from the communication interface 30 to the external source with an identifier uniquely identifying the device 12 and/or the sensing module 10. The identifier may help ensure patient privacy because the data is associated with a particular device 12 and/or a particular sensing module 10 rather than with a particular patient, and/or can allow the device 12 to be identified as an authentic device authorized to gather and transmit data to the receiver of the data. The external source that receives the data from the communication interface 30 can be configured to identify the patient with which the identifier is associated, such as by accessing a stored lookup table correlating particular patients with particular identifiers for each of a plurality of drug delivery devices and/or sensing modules. The identifier can have a variety of configurations, e.g., numeric, alphanumeric, etc. In an exemplary embodiment, the identifier is an identification code of the device 12 as reflected on a bar code attached to the device 12, which drug delivery devices often have for tracking purposes. The bar code can be scanned with an appropriate scanner and stored in the memory 28 for transmission by the communication interface 30 in connection with sensed data. In some embodiments, a photograph can be taken of the bar code, such as with a camera of a mobile phone (or other computer system), and the image can be analyzed by the mobile phone (or other computer system) that took the picture to identify the bar code from the image.

In another exemplary embodiment, data transmission can be encrypted (e.g., an encrypted BLE transmission, etc.), and the unique identifier can be part of the encrypted transmission. The identifier being part of the data transmission allows for unique identification without a user needing to scan a bar code, take a photograph, or take another action. The data would be decrypted by the computer system that receives the data to allow for identification of the identifier. Various encryption techniques can be used, as will be appreciated by a person skilled in the art, such as by using a key-based security system, e.g., a public key/private key cryptographic system, to allow for data encryption and decryption. Public and private keys can be stored in a memory and can be generated using cryptographic algorithms. Keys can be used to encrypt data for transmission and to decrypt encrypted data received from a different computing device. In such systems, a public key associated with the intended receiver of the data can be utilized to encrypt data, however, only the recipient's private key can be used to decrypt the encrypted data. In at least some embodiments, a cryptographic system such as a public key infrastructure (PM), in which one or more third parties, known as “certificate authorities,” can be used to certify ownership of the public and private key pairs. Examples of key-based security systems include the Diffie-Hellman key exchange protocol, the Digital Signature Standard (DSS) protocol, password-authenticated key agreement protocols, the Rivest-Shamir-Adelman (RSA) encryption algorithm, the Cramer-Shoup cryptosystem, and the YAK authenticated key agreement protocol. Any type of encryption (including Wired Equivalent Privacy (WEP), Wi-Fi Protected Access (WPA), Wi-Fi Protected Access II (WPA2), and Wi-Fi Protected Access III (WPA3) encryption methods) can be used to encrypt transmitted data. Various digital certificate validation schemes and cryptographic protocols, including the Secure Sockets Layer protocol (SSL), the Transport Layer Security protocol (TLS), RSA, or any other public/private key protocols can be utilized in establishing the communication.

In addition to or instead of transmitting encrypted data for identifier purposes, any other transmitted data described herein can be encrypted to improve security.

As mentioned above, the sensor 26 can be configured to sense any one or more of a variety of parameters, such as date, time, vibration, temperature, sound, motion, humidity, pressure, fluid level, force, location, proximity, and spatial orientation. As will be appreciated by a person skilled in the art, the sensor 26 can include one sensor configured to sense all of the parameter(s) being sensed by the sensing module 10, or the sensor 26 can include two or more sensors each configured to sense one or more of the parameters being sensed by the sensing module 10. In embodiments in which the sensor 26 includes multiple sensors, each of the sensors can be configured to sense different parameter(s) from one another, which may maximize a number of parameters that the sensing module 10 can sense. In embodiments in which multiple parameters are sensed, a combination of sensed parameters can be used to confirm proper drug delivery device 12 operation, e.g., by using each of sound and motion to determine when the drug delivery process has begun and/or for detecting when drug delivery has been completed.

Examples of a sensor 26 configured to gather date data and/or time data include a clock generator and a timer. The sensor 26 being configured to gather date data and/or time data may allow for other sensed parameter(s) to be accurately date and/or time stamped. The date/time stamping can thus facilitate identification of when the drug 14 was delivered from the device 12 as indicated by one or more other parameter(s) sensed by the sensor 26, as discussed further below. Similarly, date/time stamping can facilitate a determination that the drug 14 was not delivered on schedule, e.g., if the one or more other parameter(s) sensed by the sensor 26 at an expected date/time or in an expected date/time range are not indicative of the drug 14 being delivered from the device 12. In this way, sensing date and/or time may facilitate evaluation of patient compliance with a predetermined drug delivery schedule and/or evaluation of a patient's condition based on how often and/or when the drug is being administered to the patient on demand.

Examples of a sensor 26 configured to gather vibration data include an accelerometer and a motion sensor. The sensor 26 being configured to gather vibration data may allow for detecting when the drug delivery process has begun and/or for detecting when drug delivery has been completed. Vibration of an autoinjector such as the device 12 is indicative of a spring (obscured in FIG. 1) disposed within the housing 18 and operatively coupled to the needle shield 20 being actuated to cause the needle to be advanced after the needle shield 20 has moved in response to being pressed against a skin surface. Similarly, vibration of an autoinjector such as the device 12 is indicative of another spring (obscured in FIG. 1) disposed within the housing 18 causing the needle shield 20 to move to a locked position after the drug 14 has been delivered. Knowing that drug delivery has occurred (with or without being time/date stamped) may facilitate compliance analysis because it can be known whether a dose was delivered from the device 12. Vibration data indicative of a desired action (e.g., start of drug delivery, end of drug delivery, shaking of the device 12 for drug mixing purposes, etc.) can be distinguished from other vibration data that may be gathered by the sensor 26 due to, e.g., transport of the device 12, removing the device 12 from packaging, bumping of the device 12 against something, etc. As will be appreciated by a person skilled in the art, an algorithm can allow for differentiation between different signals, such as by using a fast Fourier transform (FFT) to analyze a frequency spectrum of gathered vibration data. Correlating vibration data with a date/time may allow for more precise compliance analysis and/or may facilitate a determination that the drug 14 was delivered properly. For example, a certain, known amount of time (or range of time) may be expected to pass between the start of the drug delivery process and the end of the drug delivery process. If the vibration data indicates that too little time or too much time passes between the start of the drug delivery process and the end of the drug delivery process, improper drug delivery may have occurred and may be flagged for follow-up by a medical professional with the user. For another example, if only one vibration event is detected as happening, then the drug delivery process may have begun (as indicated by first vibration data) but not ended properly (as indicated by a lack of second vibration data following the first vibration data) due to device malfunction, such as by the needle not being retracted and/or the needle shield not extending over the needle.

Examples of a sensor 26 configured to gather temperature data include a temperature sensor (e.g., a thermistor, a thermocoupler, etc.). The sensor 26 being configured to gather temperature data (e.g., ambient temperature data) may provide information as to whether the drug 14 is at a safe temperature for storage and/or for delivery to a user, as a safe temperature (or safe temperature range) for the drug 14 is a known value. The sensor 26 being configured to gather temperature data (e.g., ambient temperature data) may provide information helpful for instances in which the sensing module 10 is being used in a clinical trial since monitoring temperature may help make sure the drug didn't undergo a temperature excursion that would throw off the clinical data. Correlating temperature data with a date/time may facilitate analysis of the patient's treatment using the drug 14 since the drug 14 being delivered when at or previously at an improper temperature may adversely affect the drug's efficacy.

Examples of a sensor 26 configured to gather sound (acoustic) data include an acoustic sensor, a microphone, and an accelerometer. The sensor 26 being configured to gather sound data may allow for detecting when the drug delivery process has begun and/or for detecting when drug delivery has been completed. Acoustic data can be indicative of when drug delivery has begun, e.g., by detected sound being indicative of the needle of the device 12 being advanced through spring or other mechanical action by being data within a predetermined frequency range and/or of a particular duration, by detected sound being indicative of a cap (not shown) being removed from the needle shield 20 by being data within a predetermined frequency range and/or of a particular duration, by detected sound (e.g., a “click” noise or other noise created by mechanical part(s)) being indicative of a trigger of a drug delivery device being manually depressed, by detected sound being indicative of a malfunction, etc. Similarly, acoustic data can be indicative of when drug delivery has stopped, e.g., by detected sound being indicative of the needle of the device 12 being retracted through spring or other mechanical action by being data within a predetermined frequency range and/or of a particular duration, by detected sound being indicative of the needle shield 20 advancing over the needle by being data within a predetermined frequency range and/or of a particular duration, by detected sound being indicative of a piston of the device 12 stopping movement through the container 16 to displace the drug 14 through the needle by being data within a predetermined frequency range and/or of a particular duration, by detected sound being indicative of a trigger of a drug delivery device being manually released, etc. Correlating sound data with a date/time may allow for more precise compliance analysis and/or may facilitate a determination that the drug 14 was delivered properly similar to that discussed above regarding vibration data.

Examples of a sensor 26 configured to gather motion data include a motion sensor, an accelerometer, a micro switch, a capacitive switch, an optical position switch, and a magnetic sensor. The sensor 26 being configured to gather motion data may allow for detecting when the drug delivery process has begun, for detecting when drug delivery has been completed, and/or for detecting premature removal of the device 12 from a patient before completion of drug delivery. The device 12 moving from a still state to a state of movement, as detected by the motion sensor, may be indicative of a possible start of a drug delivery process, e.g., by the device 12 being picked up by a user. If after the start of injection has been sensed, the sensor 26 detects motion prior to the sensor 26 sensing end of drug delivery and within a predetermined expected duration of drug delivery, it can be determined that the device 12 was lifted too early and that drug delivery therefore likely did not properly complete. The sensor 26 being configured to gather motion data may allow for detecting an orientation of the drug delivery device 12 during drug delivery to allow for determining whether the device 12 was in a proper orientation for injection, such as a proper position of an injector being in a vertical, substantially perpendicular orientation relative to the patient's skin versus an improper position of being at a non-perpendicular angle relative to the patient's skin. A date/time stamp of detected motion can be correlated with one or more other date/time stamped sensed parameters to determine whether the detected motion is actually indicative of the start of the drug delivery process as opposed to other motion, such as the device 12 being transported by a user. Correlating motion data with a date/time may thus allow for more precise compliance analysis and/or may facilitate a determination that the drug 14 was delivered properly similar to that discussed above regarding vibration data.

Examples of a sensor 26 configured to gather humidity data include a thermistor, a humistor, and a hygrometer. The sensor 26 being configured to gather humidity data may provide information as to whether the drug 14 is at a safe humidity level for storage and/or for delivery to a user, as a safe humidity level (or safe humidity level range) for the drug 14 is a known value. The sensor 26 being configured to gather humidity data may provide information helpful for instances in which the sensing module 10 is being used in a clinical trial since monitoring humidity may help make sure the drug didn't undergo a humidity excursion that would throw off the clinical data. Correlating humidity data with a date/time may facilitate analysis of the patient's treatment using the drug 14 similar to that discussed above regarding temperature.

Examples of a sensor 26 configured to gather pressure data include a pressure sensor and a Hall effect sensor. The sensor 26 being configured to gather pressure data may allow for detecting when drug delivery has begun by the sensing module 10 being positioned on the device 12 at a location where a user is likely to hold the device 12 for drug delivery. Thus, pressure on the sensing module 10 as detected by the pressure sensor can be indicative of the device 12 being held at a start of the drug delivery process. Similarly, pressure decreasing as detected by the pressure sensor can be indicative of the device 12 being released at an end of the drug delivery process. The sensor 26 being configured to gather pressure data may allow for detecting an altitude at which the drug delivery device 12 is located, as pressure (absolute or relative) can indicate an elevation above sea level. Different drugs may flow or perform differently at different altitudes, which gathered pressure data may allow to be identified. A date/time stamp of detected pressure can be correlated with one or more other date/time stamped sensed parameters to determine whether the detected pressure is actually indicative of the start of the drug delivery process as opposed to other pressure, such as the device 12 being transported by a user. Correlating pressure data with a date/time may allow for more precise compliance analysis and/or may facilitate a determination that the drug 14 was delivered properly similar to that discussed above regarding vibration data.

Examples of a sensor 26 configured to gather fluid level data include a non-contact water level switch (e.g., Doppler). The sensing device 10 in this illustrated embodiment is not attached to the device 12 at a position where fluid level of the drug 14 in the container 16 can be accurately gathered, but in other embodiments, a sensing device could be positioned relative to a container so as to be configured to accurately gather data regarding a level of fluid in the container.

Examples of a sensor 26 configured to gather force data include a forge gauge and a flexible force sensor. The sensor 26 being configured to gather force data may allow for detecting whether the device 12 is being held with sufficient force against the patient's skin during injection (which may be detected using one or more types of parameter data as discussed herein), which may help detect or explain injection failure if inadequate force was detected as compared to a predetermined force threshold that is known for proper injection.

Examples of a sensor 26 configured to gather location data include a location sensor such as a global positioning satellite (GPS) sensor. The location sensor can be part of a device already associated with the patient, such as a smart phone with location sensing capability. The sensor 26 being configured to gather location data may allow for other sensed parameter(s) to be accurately location stamped. The location stamping can thus facilitate identification of a geographic location where the drug 14 was delivered from the device 12 as indicated by one or more other parameter(s) sensed by the sensor 12, as discussed further below. In this way, sensing location may facilitate evaluation of patient compliance with a predetermined drug delivery schedule, e.g., by allowing identification of locations where the patient is missing scheduled dose(s) and receiving scheduled dose(s).

Examples of a sensor 26 configured to gather proximity data include a proximity sensor (e.g., an optical sensor, a Hall effect sensor, etc.). The sensor 26 being configured to gather proximity data may allow for detecting that the device 12 is being held against skin when drug delivery begins, such as with an autoinjector or other injection device that is held against skin during drug delivery. If at a time/date of when a start of injection has been sensed, the sensor 26 detects a distance of the device 12 from skin that is above a predetermined threshold distance (or that is outside of a predetermined threshold distance range), it can be determined that the device 12 was not being held against skin when drug delivery started and that drug delivery therefore was not properly performed and/or that the full dose of drug was likely not delivered to the patient. The sensor 26 is at a known location on the drug delivery device 12 such that the sensor 26 will have a known distance from skin when the device 12 is being held properly against a skin surface for drug delivery, e.g., when the device 12 is being held normal to a skin surface. The predetermined threshold distance (or predetermined threshold distance range, which may account for one or more factors such as manufacturing tolerances) can thus be based on the known distance of the sensor 26 from skin. The sensor 26 being configured to gather proximity data may allow for detecting premature removal of the device 12 from a patient, e.g., from a skin surface of the patient such as with an autoinjector or other injection device, before completion of drug delivery. If after a start of injection has been sensed and before the end of injection has been sensed, the sensor 26 detects a distance of the device 12 from skin that is above a predetermined threshold distance (or that is outside of a predetermined threshold distance range), it can be determined that the device 12 was lifted too early, e.g., as indicated by the distance of the device 12 from skin being too high, and that drug delivery therefore likely did not properly complete. The sensor 26 being configured to gather proximity data may allow for confirming an end of drug delivery. In some instances it may be difficult to differentiate between an end of drug delivery and occurrence of another event that occurs very near the end of drug delivery. For example, a needle shield of an autoinjector can be deployed very near the end of drug delivery, such as the needle shield being automatically deployed in response to the drug delivery device being lifted up and removed from the patient's skin. A first spring of the autoinjector can cause the sensor 26 that includes a first sound sensor to detect a sound when injection of the drug is complete due to the first spring's involvement in needle deployment, and a second spring of the autoinjector cause the sensor 26 that includes a sound sensor to detect a second sound when injection has ended due to the second spring's involvement in deploying the needle shield when the autoinjector is lifted from skin. The first and second sounds can be close enough in time that it may be difficult to determine which of the first and second sounds began first. The proximity data can be used in combination with the sound data to determine whether the autoinjector was lifted from skin before drug delivery was complete, e.g., by allowing date/time stamped proximity data to be correlated with date/time stamped sound data.

Examples of a sensor 26 configured to gather spatial orientation data include an accelerometer, a tilt/angle switch (mercury free), and a position sensor. The sensor 26 being configured to gather spatial orientation data may allow for detecting the drug delivery device's orientation relative to ground. A particular spatial orientation of the device 12 may be known to correspond to a drug delivery position of the device 12, e.g., when the device 12 is being held normal to a skin surface. Correlating spatial orientation data with a date/time may allow for more precise compliance analysis and/or may facilitate a determination that the drug 14 was delivered properly similar to that discussed above regarding vibration data. Correlating spatial orientation data with sound data and/or proximity data may allow for more precise compliance analysis and/or may facilitate a determination that the drug 14 was delivered properly, e.g., determining whether the full dose of the drug was injected (or otherwise delivered) and/or whether the drug delivery device was in the correct orientation when the drug was delivered (such as by being in a vertical, substantially perpendicular orientation relative to the patient's skin versus being at a non-perpendicular angle relative to the patient's skin).

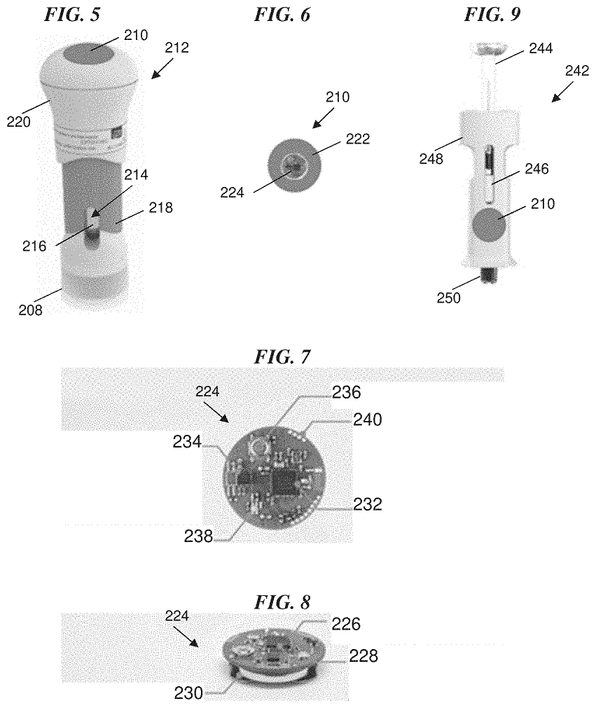

FIGS. 5 and 6 illustrate another embodiment of a sensing module 210 configured to gather data for one or more parameters related to drug delivery and to transmit data indicative of the gathered data to an external source configured to analyze the data received from the sensing module 210. FIG. 6 illustrates the sensing module 210 as a standalone element. The sensing module 210 is generally configured and used similar to the sensing module 10 of FIGS. 1 and 2. FIG. 5 illustrates the sensing module 210 attached to another embodiment of a drug delivery device 212 configured to deliver a drug 214, which is a clear liquid in this illustrated embodiment. The drug delivery device 212 in this illustrated embodiment is an autoinjector configured to inject the drug 214 from a container 216 in a housing 218 of the device 212 and out of a needle (obscured in FIG. 5) in response to manual depressing of a head 220 of the device 212 relative to the housing 218. The drug delivery device 212 also includes a removable cap 208 configured to be removed from a remainder of the device 212 by a user to expose a needle shield of the device 212.

The sensing module 210 is attached non-removably to an outer surface of the device 212, although as mentioned above the sensing module 210 can instead be removably attached to the device 212. The outer surface is an outer surface of the depressible head 220, but the sensing module 210 can be attachable to another outer surface of the drug delivery device 210, such as on the housing 218, etc. The sensing module 210 can be attached anywhere on the head 220 but in this illustrated embodiment is attached to a top surface of the head 220. The top surface of the head 220 is where a user typically applies pressure to the head 220 to depress the head 220 and cause drug delivery. The sensing module 210 is attached to the device 212 with an adhesive, e.g., a layer of an adhesive on the sensing module 210, in this illustrated embodiment, but the sensing module 210 can be attached to a drug delivery device in other ways, as discussed above.

The sensing module 210 includes a variety of electronic components to facilitate the gathering of data and the transmission of gathered data to an external source similar to that discussed above regarding the sensing module 10 of FIGS. 1 and 2. FIGS. 7 and 8 illustrate one embodiment of a PCB 224 supporting the sensing module's electronic components. The PCB 224 is a MetaWear sensor available from MbientLab of San Francisco, Calif. FIG. 6 illustrates the PCB 224 attached to an underside of a base 222 configured to have the layer of adhesive thereon surrounding the PCB 224.

As shown in FIGS. 7 and 8, the PCB 224 is rigid and includes a processor 226, a memory 228, a power source 230 in the form of a coin cell battery, a communication interface 232 configured to communicate using BLE, a motion sensor 234, a pressure sensor 236 in the form of a push button, and an LED 238. The PCB 224 also includes free real estate 240 for one or more additional sensors. The sensing module 210 being attached to the top surface of the head 220 allows the pressure sensor 236 to be pressed on when the head 220 is depressed manually by a user and for the pressure on the pressure sensor 236 to be released when the user removes pressure from the head 220. The motion sensor 234 being on the head 220 facilitates motion being used as an indicator of the start of drug delivery since the head 220 is moved from its resting position (shown in FIG. 5) by being pressed down in a direction toward and relative to the housing 218 to start the drug delivery process. The motion sensor 234 being on the head 220 also facilitates motion being used as an indicator of the end of drug delivery since the head 220 stops moving relative to the housing 218 when the drug delivery process has ended.

FIG. 9 illustrates another embodiment of a drug delivery device 242 with the sensing module 210 of FIG. 6 attached thereto. The drug delivery device 242 in this illustrated embodiment is a safety syringe contained in a removable grip accessory 248 and is configured to inject a drug (obscured in FIG. 9) from a barrel 246 of the device 242 and out of a needle (obscured by a needle shield 250 of the grip accessory 248 in FIG. 9) in response to manual depressing of a plunger 244 of the device 242.

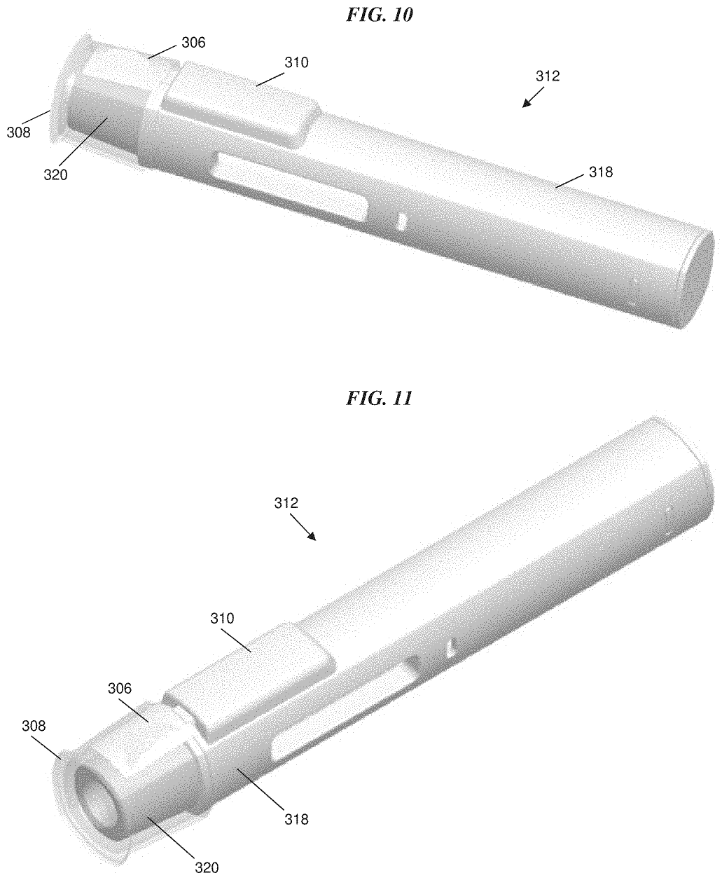

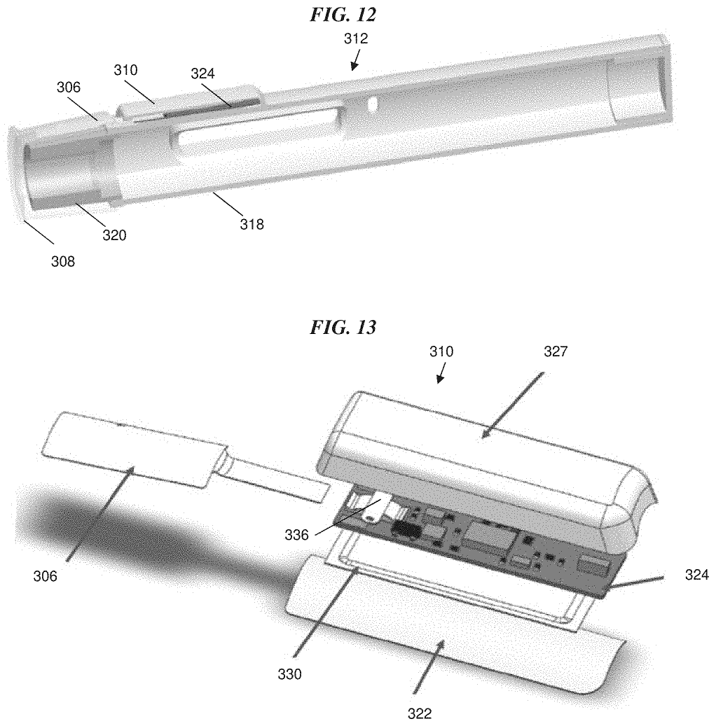

FIGS. 10-13 illustrate another embodiment of a sensing module 310 configured to gather data for one or more parameters related to drug delivery and to transmit data indicative of the gathered data to an external source configured to analyze the data received from the sensing module 310. FIG. 13 illustrates the sensing module 310 as a standalone element. The sensing module 310 is generally configured and used similar to the sensing module 10 of FIGS. 1 and 2. FIGS. 10-12 illustrate the sensing module 310 attached to another embodiment of a drug delivery device 312 configured to deliver a drug (not shown). The drug delivery device 312 in this illustrated embodiment is an autoinjector configured to inject the drug from a container (not shown) in a housing 318 of the device 312 and out of a needle (not shown) in response to a needle shield 320 of the device 312 moving upward toward and into the housing 318, e.g., by the needle shield 320 being pressed against a patient's skin. The drug delivery device 312 also includes a removable cap 308 configured to be removed from a remainder of the device 312 by a user to expose the needle shield 320.

The sensing module 310 is attached non-removably to an outer surface of the device 312, although as mentioned above the sensing module 310 can instead be removably attached to the device 312. The outer surface is an outer surface of the housing 318, but the sensing module 310 can be attachable to another outer surface of the drug delivery device 310 as discussed herein. The sensing module 310 can be attached anywhere on the housing 318 but in this illustrated embodiment is attached adjacent to the cap 308 to facilitate use of a tab 306 as a tamper resistant feature, as discussed further below. The sensing module 310 is attached to the device 312 with an adhesive, e.g., a layer of an adhesive on a bottom portion 322 of a housing of the sensing module 310. However, as discussed herein, the sensing module 310 can be attached to a drug delivery device in other ways.

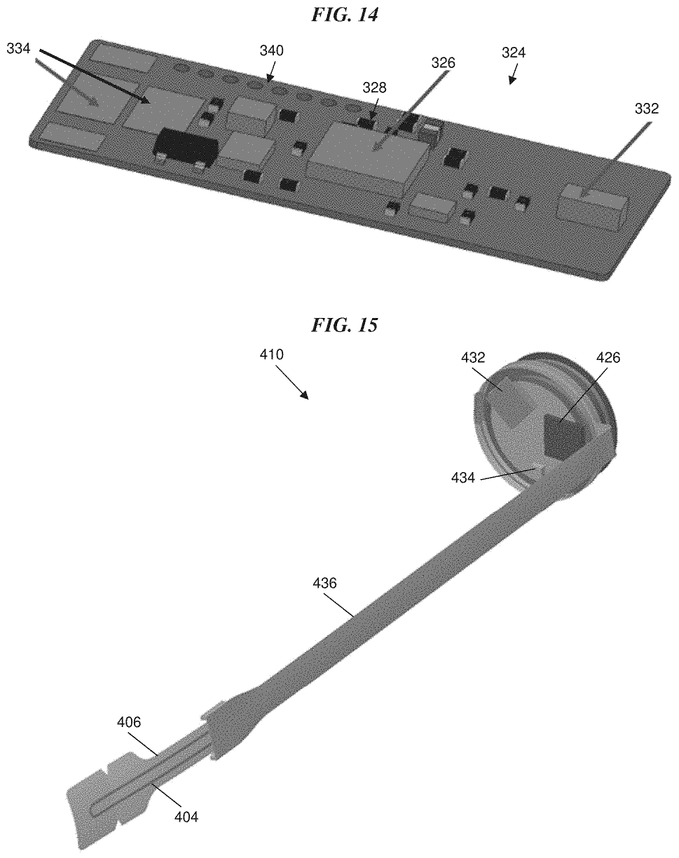

The sensing module 310 includes a variety of electronic components to facilitate the gathering of data and the transmission of gathered data to an external source similar to that discussed above regarding the sensing module 10 of FIGS. 1 and 2. The sensing module 310 includes a housing defined by the bottom housing portion 322 and a top housing portion 327 that are fixed together. A PCB 324, shown in FIGS. 13 and 14, is disposed in the housing and supports the sensing module's electronic components. The PCB 324 in this illustrated embodiment is rigid, although as mentioned above may instead be flexible. The PCB 324 includes a processor 326, a memory 328, a communication interface 332 in the form of a chip antenna, switch contact pads 334, a switch 336, and free real estate 340 for one or more sensors.

A power source 330 is disposed within the housing and is configured to selectively provide power to one or more of the sensing module's electronic components, e.g., the processor 326, sensor(s), etc. The power source 330 being configured to selectively provide power may help ensure that the power source 330 is not depleted of power before the drug is injected from the device 312 (e.g., because of a length of time the device 312 was stored before use) and/or may allow the power source 330 to be relatively small and/or inexpensive since power only need be provided for a relatively short duration of time during one-time use of the device 312 for drug delivery. The power source 330 is configured to not provide power when the tab 306 is coupled to the sensing module 310 and is configured to provide power when the tab 306 is not coupled to the sensing module 310. The tab 306 is configured to move from a first position, in which the tab 306 is coupled to the sensing module 310 (corresponding to the power source 330 not providing power), to a second position, in which the tab 306 is not coupled to the sensing module 310 (corresponding to the power source 330 providing power). With the tab 306 in the first position, as shown in FIGS. 10-12, the tab 306 acts as an insulator to prevent the switch 336 from engaging the switch contact pads 334 (FIG. 14), thereby creating an open circuit that prevents the power source 330 from providing power to electronic components of the sensing module 310. The electronic components are thus “off” as a result of not receiving power. The tab 306 is made from an insulating material, such as Mylar® or non-conductive, insulating material, to allow the tab 306 to act as an insulator. With the tab 306 in the second position, the switch 336 is allowed to engage the switch contact pads 334, thereby creating a closed circuit that allows the power source 330 to provide power to electronic components of the sensing module 310. The tab 306 is thus configured to “wake up” the sensing module 310 by moving from the first position to the second position. The sensing module's power source 330 may therefore not run out of power before the end of drug delivery since power will not begin being used until the tab 306 is removed, e.g., the power source 330 has zero shelf life power consumption.

The tab 306 can have a variety of sizes, shapes, and configurations. In this illustrated embodiment, the tab 306 has a first, lower portion located outside of the sensing module 310 and attached to the cap 308, such as by being adhered thereto with adhesive or other attachment mechanism. The tab 306 has a second, upper portion extending from the first portion and extending into the sensing module 310, e.g., into the housing of the sensing module 310. The second portion of the tab 306 is positioned so as to prevent the switch 336 from engaging the switch contact pads 334. In this way, when the tab 306 is removed from the sensing module 310 and is no longer located within the housing 318, the tab 306 no longer prevents the switch 336 from engaging the switch contact pads 334, e.g., closing the open circuit that exists when the tab 306 is in the first position.

The tab 306 being attached to the cap 308 facilitates movement of the tab 306 from the first position to the second position. When the cap 308 is manually removed by a user from a remainder of the drug delivery device 312, the tab 306 attached thereto is also removed from the remainder of the drug delivery device 312, thereby also de-coupling the tab 306 from the sensing module 310 that is attached to the drug delivery device 312. The tab 306 is thus configured to move from the first position to the second position in response to removal of the cap 308. A user therefore need not take any special action to activate the power source 330, e.g., cause the power source 330 to start providing power, since cap 308 removal is a normal part of using the device 312. In other words, when the cap 308 is pulled off the housing 318, the tab 306 is pulled out of the sensing module 310 to move from the first position to the second position.

As in this illustrated embodiment, the tab 306 can be configured as a tamper resistant feature. The tab 306 being absent but the cap 308 being on the drug delivery device 308 may be evidence of tampering, e.g., evidence that the cap 308 was removed at some prior time and then replaced back on the device 312. Similarly, the tab 306 being attached to the cap 308 without the tab's second portion located in the housing of the sensing module 310 may be indicative of tampering, evidence that the cap 308 was removed at some prior time and then replaced back on the device 312.

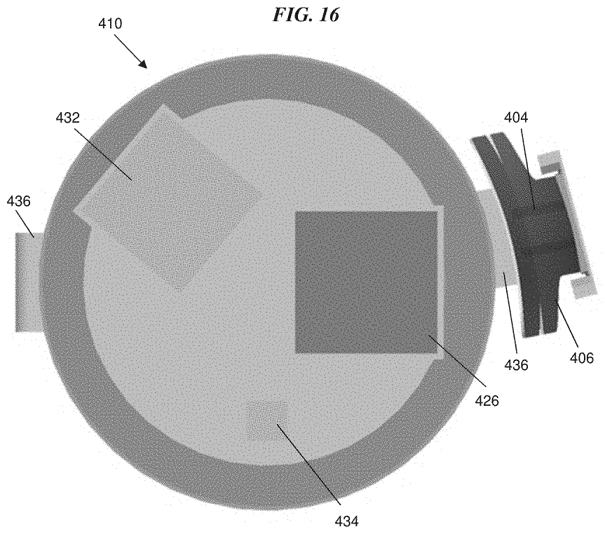

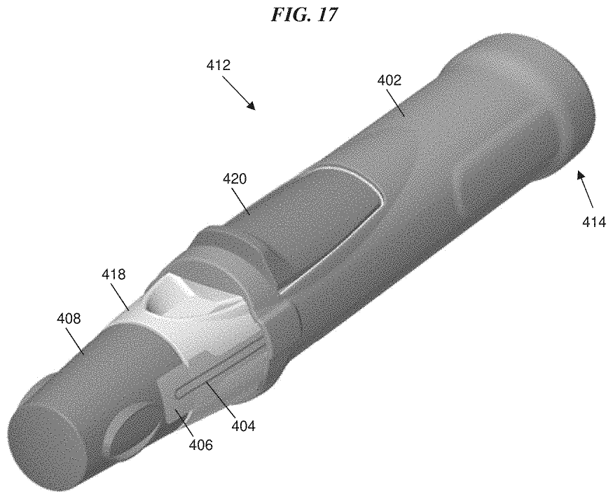

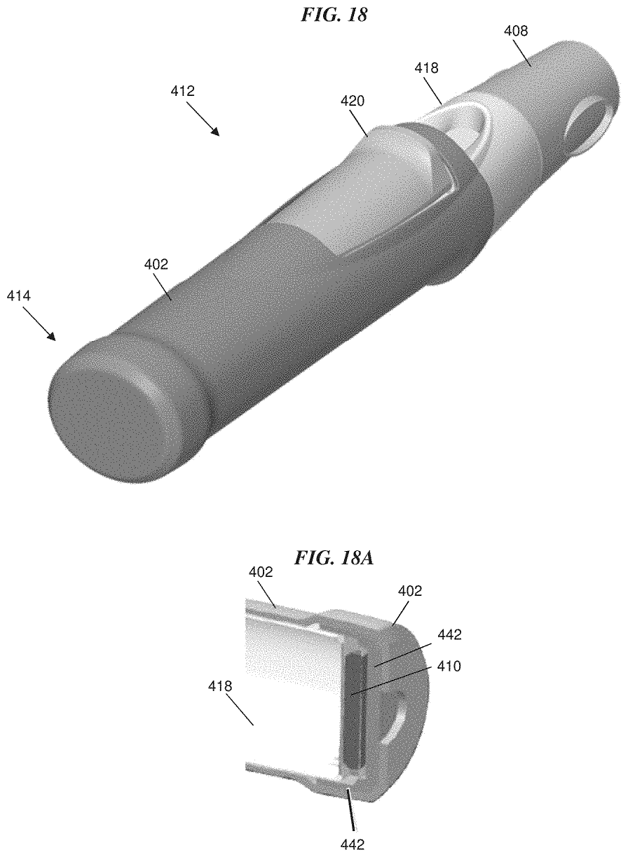

FIGS. 15 and 16 illustrate another embodiment of a sensing module 410 configured to gather data for one or more parameters related to drug delivery and to transmit data indicative of the gathered data to an external source configured to analyze the data received from the sensing module 410. FIGS. 15 and 16 illustrate the sensing module 410 as a standalone element. The sensing module 410 is generally configured and used similar to the sensing module 10 of FIGS. 1 and 2. FIGS. 17 and 18 show another embodiment of a drug delivery device 412 configured to deliver a drug (obscured in FIGS. 17 and 18) and having the sensing module 410 attached thereto. The drug delivery device 412 in this illustrated embodiment is an autoinjector configured to inject the drug from a container (obscured in FIGS. 17 and 18) in a housing 418 of the device 412 and out of a needle (not shown) in response to manual actuation by a user of a trigger 420 of the device 412. The drug delivery device 412 also includes a removable cap 408 configured to be removed from a remainder of the device 412 by a user to expose the needle.

The sensing module 410 is obscured in FIGS. 17 and 18 because the sensing module 410 is on an outer surface of the housing 418 but is disposed under an outer boot 402 of the drug delivery device 412. The sensing module 410 being disposed under the outer boot 402 of the drug delivery device 412 may help protect the sensing module 410 from damage by, e.g., helping to prevent the sensing module 410 from coming into contact with liquid, providing a protective layer over the sensing module 410 that may provide protection in the event that the device 412 is dropped, etc. The outer boot 402 is rubber in this illustrated embodiment, which may facilitate user gripping of the device 412, ease attachment of the outer boot 402 to the housing 418 easier by stretching over the housing 418, ensure a secure connection to the housing 418 by stretching to accommodate the size and shape of the housing 418, and/or enhance crash protection of the sensing module 410, but the outer boot 402 can be made of other materials. The sensing module 410 being disposed within the outer boot 402 may facilitate attachment of the sensing module 410 to an outer surface of the housing 418. After the drug delivery device 412 is otherwise manufactured, the sensing module 410 can be positioned outside the housing 418 and coupled to the housing 418 by the outer boot 402 being placed over the sensing module 410. In other embodiments, the sensing module 410 can be located on an exterior surface of the outer boot 402, which may facilitate retrofitting of the sensing module 410 onto an existing drug delivery device, and/or can be located in the outer boot 402, e.g., embedded therein.

The sensing module 410 includes a variety of electronic components to facilitate the gathering of data and the transmission of gathered data to an external source similar to that discussed above regarding the sensing module 10 of FIGS. 1 and 2. The electronic components are located at an end 414 of the drug delivery device 412 that is opposite to the end with the cap 408. The sensing module 410 includes a PCB 424 that includes a processor 426, a memory (not shown), a communication interface 432 in the form of a Bluetooth module, and a sensor 434. As shown in FIGS. 15, 16, and 19, the sensing module 410 also includes a receiver coil 440 configured to facilitate communication using the communication interface 432, a power source 430 in the form of a coin cell battery, and a flexible circuit 436. The receiver coil 440 can also facilitate wireless charging of the power source 430. The circuit 436 being flexible may facilitate smooth, close positioning of the circuit 436 along a longitudinal length of an outer surface of the housing 418 (as shown in FIG. 17), which may be curved or have surface features thereon that the flexibility may accommodate. In other embodiments, however, the circuit 436 can be rigid.

As shown in FIG. 18A, an end cap 442 is positioned over the portion of the sensing module 410 at the end 414 of the device 412. The end cap 442 is configured to provide protection for the sensing module 410.

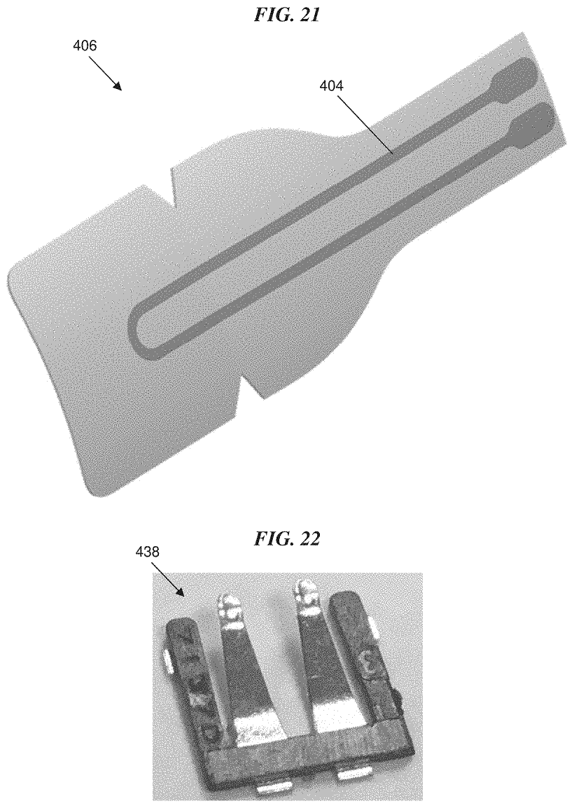

The power source 430 is configured to selectively provide power to one or more of the sensing module's electronic components, e.g., the processor 426, the communication interface 432, the sensor 434, etc., similar to that discussed above regarding the power source 330 of FIG. 13. The power source 430 is configured to not provide power when a tab 406 is coupled to the sensing module 410 and is configured to provide power when the tab 406 is torn or is not coupled to the sensing module 410. The tab 406 is shown coupled to the sensing module 410 in FIGS. 15-17 and 20 (and FIG. 18, although the tab 406 is obscured in FIG. 18) and is shown as a standalone element in FIG. 21. The tab 406 is configured to move from a first position, in which the tab 406 is coupled to the sensing module 410 (corresponding to the power source 430 not providing power), to a second position, in which the tab 406 is torn or is not coupled to the sensing module 410 (corresponding to the power source 430 providing power). With the tab 406 in the first position, the tab 406 prevents the flexible circuit 436 from electrically connecting the power source 430 with the electronic components of the PCB 424 by interrupting current flow, which prevents the power source 430 from providing power to the electronic components of the PCB 424. With the tab 406 in the second position, the flexible circuit 436 electrically connects the power source 430 with the electronic components of the PCB 424 by allowing current flow, which allows the power source 430 to provide power to the electronic components of the PCB 424. The tab 406 is thus configured to “wake up” the sensing module 410 by moving from the first position to the second position. The sensing module's power source 430 may therefore not run out of power before the end of drug delivery since power will not begin being used until the tab 406 is removed, e.g., the power source 430 has zero shelf life power consumption.

Similar to that discussed above regarding the tab 306 of FIG. 13, the tab 406 is configured to be removed from the sensing module 410, e.g., to move from the first position to the second position, in response to removal of the cap 408 by a user. The tab 406 has a first, lower portion located outside of the sensing module 410 and attached to the cap 408, such as by being adhered thereto with adhesive and/or other attachment mechanism. The tab 406 has a second, upper portion extending from the first portion and extending into contact with the flexible circuit 436 of the sensing module 410. The second portion of the tab 406 extends along an outer surface of the housing 418. The second portion of the tab 406 is attached to the outer surface of the housing 418 with an adhesive and/or other attachment mechanism. In this illustrated embodiment, when the cap 408 is pulled off the housing 418, the tab 406 is configured to tear, e.g., at a junction between the first and second portions of the tab 406, to move from the first position to the second position.

As shown in FIGS. 15-17, 20, and 21, the tab 406 includes a conductive trace 404 thereon. The conductive trace 404 can be provided in a variety of ways, such as with conductive ink or conductive tape. In this illustrated embodiment the conductive trace 404 is formed with conductive ink printed on the tab 406, which can be paper or other material. With the tab 406 in the first position, the conductive trace 404 is coupled to the flexible circuit 436 of the sensing module 710 to form a circuit that prevents the flexible circuit 436 from electrically connecting the power source 430 to the PCB 424, e.g., by a pin of the sensing module's processor reading zero volts due to the tab 406 interrupting current flow such that the processor is not receiving electrical power and thus cannot register any voltage. With the tab 406 in the second position, the conductive trace 404 is not coupled to flexible circuit 436, so the conductive trace 404 no longer forms a circuit with the flexible circuit 436. The flexible circuit 436 is thereby allowed to complete a circuit between the power source 430 and the PCB 424, e.g., by the pin of the sensing module's processor reading non-zero volts (e.g., one volt), to allow the power source 430 to start providing power to the electronic components of the PCB 424. As shown in FIG. 20, a connector 438 is provided to facilitate electrical connection between the flexible circuit 436 and the conductive trace 404 when the tab 406 is in the first position. The connector 438 is shown as a standalone element in FIG. 22. As shown in FIG. 22, the connector 438 includes two conductive terminals, which connect to the flexible circuit 436 and that are connected together by the conductive trace 404 until the tab 406 is torn or de-coupled from the sensing module 410, e.g., until the tab 406 moves from the first position to the second position.

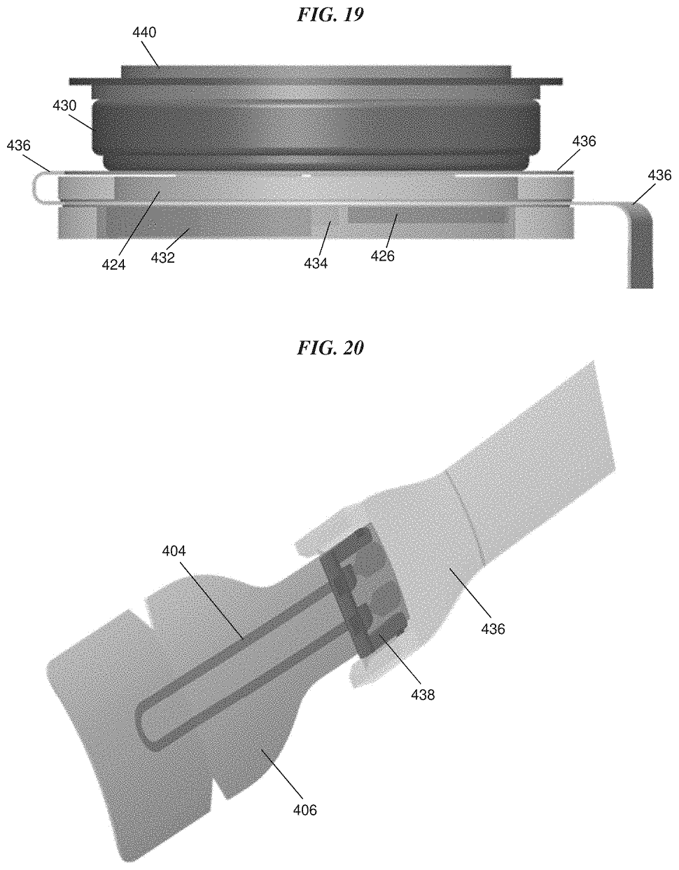

FIG. 19 illustrates one embodiment of positioning the flexible circuit 436 relative to the power source 430 and PCB 424. The flexible circuit 436 in this “two-layer” embodiment has a portion positioned between the PCB 424 and electronic components thereon and wraps around the PCB 424 to have another portion positioned between the PCB 424 and the power source 430. FIG. 23 illustrates another embodiment of positioning the flexible circuit 436 relative to the power source 430 and PCB 424. The flexible circuit 436 in this “one-layer” embodiment has a portion positioned below the electronic components on the PCB 424 and wraps around both the electronic components and the PCB 424 to have another portion positioned between the PCB 424 and the power source 430. The “one-layer” configuration or “two-layer” configuration may be easier to manufacture depending on the particular configuration of the PCB 424 and electronic components thereon.

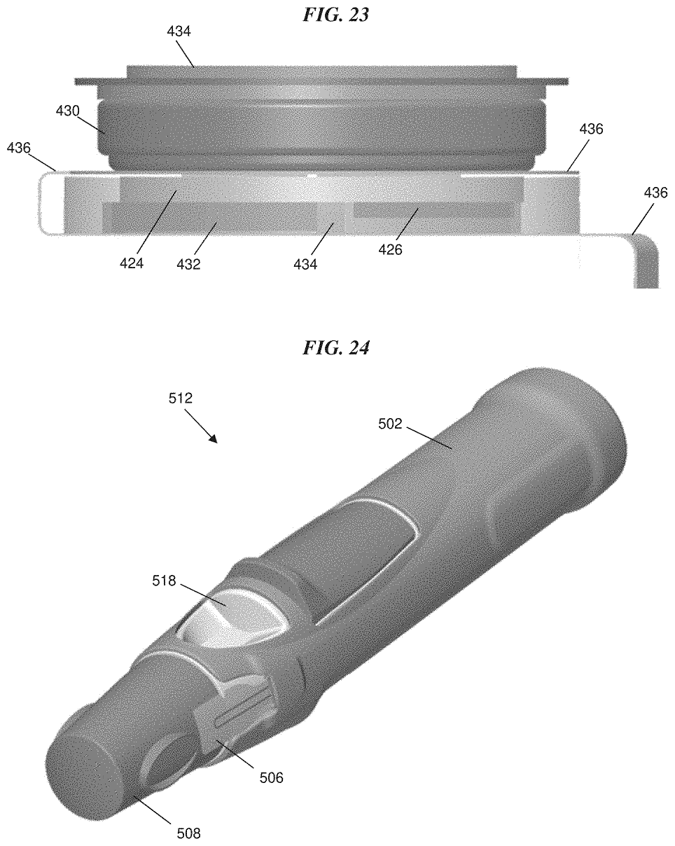

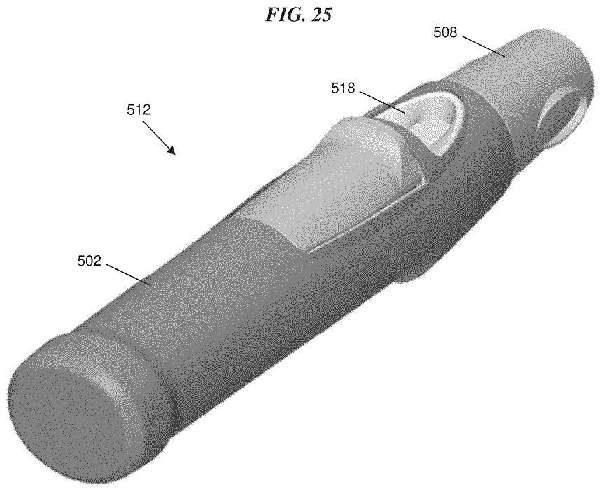

FIGS. 24 and 25 illustrate another embodiment of a drug delivery device 512 with the sensing module 410 of FIG. 15 and another embodiment of a tab 506 coupled thereto. The tab 506 is the same as the tab 406 of FIG. 15 except that the tab 506 has a shorter longitudinal length. The sensing module 410 is non-removably attached to the device 512 similar to its non-removable attachment to the drug delivery device 412 of FIGS. 17 and 18. The sensing module 410 is disposed on an outer surface of the housing 518 and under an outer boot 502 similar to its disposal under the outer boot 402 of FIGS. 17 and 18, although as mentioned above the sensing module 410 can instead be on an outer surface of the outer boot 502 or be within the outer boot 502. The device 512 of FIGS. 24 and 25 is the same as the device 412 of FIGS. 17 and 18 except that in the illustrated embodiment of FIGS. 24 and 25 the outer boot 502 has a longer longitudinal length than the outer boot 402 of FIGS. 17 and 18. The outer boot 502 of FIGS. 24 and 25 extends along an entire longitudinal length of the drug delivery device's housing 518 and terminates just proximal to the cap 508. The outer boot 502 extending along the housing's entire longitudinal length may allow for a shorter tab 506 (e.g., less longitudinal length) than may be used with an outer boot, such as the outer boot 402 of FIGS. 17 and 18, that extends along only a partial longitudinal length of the device's housing. A shorter tab 506 may facilitate de-coupling of the tab's conductive trace, e.g., from the flexible circuit 436.

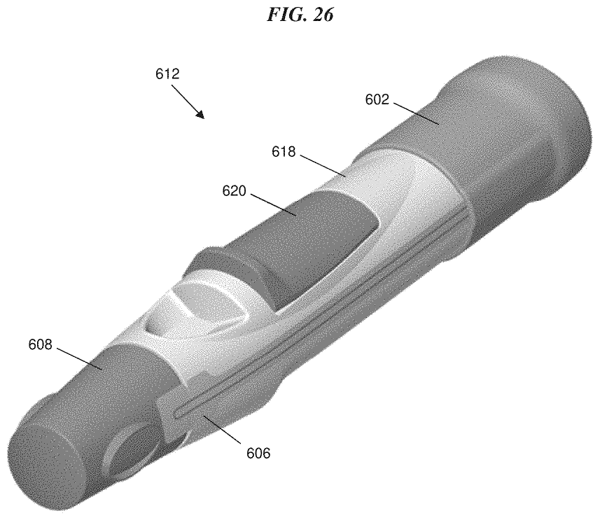

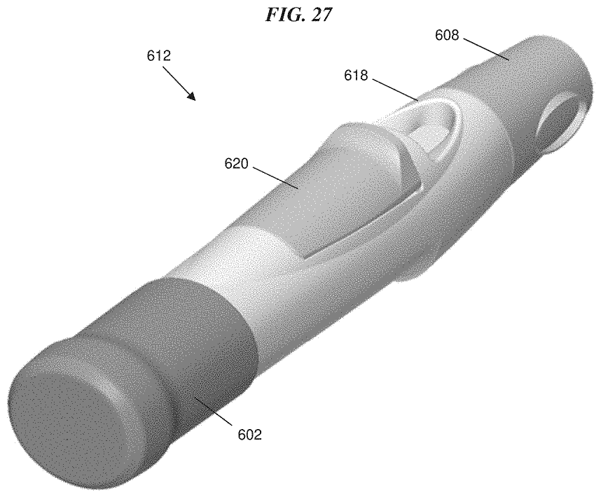

FIGS. 26 and 27 illustrate another embodiment of a drug delivery device 612 with the sensing module 410 of FIG. 15 and another embodiment of a tab 606 coupled thereto. The tab 606 is the same as the tab 406 of FIG. 15 except that the tab 606 has a longer longitudinal length. The tab 606 also has a longer length than the tab 506 of FIG. 24. The sensing module 410 is non-removably attached to the device 612 similar to its non-removable attachment to the drug delivery device 412 of FIGS. 17 and 18. The sensing module 410 is disposed on an outer surface of a housing 618 of the drug delivery device 612 and under an outer boot 602 similar to its disposal under the outer boot 402 of FIGS. 17 and 18, although as mentioned above the sensing module 410 can instead be on an outer surface of the outer boot 602 or be within the outer boot 602. The device 612 of FIGS. 26 and 27 is the same as the device 412 of FIGS. 17 and 18 except that in the illustrated embodiment of FIGS. 26 and 27 the outer boot 602 has a shorter longitudinal length than the outer boot 402 of FIGS. 17 and 18. The outer boot 602 also has a shorter longitudinal length than the outer boot 502 of FIGS. 24 and 25. The outer boot 602 of FIGS. 26 and 27 extends along only a partial longitudinal length of the drug delivery device's housing 618 and terminates proximal to the device's cap 608 and to the device's trigger 620. The outer boot 602 extending along a relatively short length of the housing's longitudinal length may allow for a shorter flexible circuit of the sensing module than may be used with an outer boot, such as the outer boots 402, 502 of FIGS. 17, 18, 24, and 25 that extend along a longer longitudinal length of the device's housing. A shorter flexible circuit and shorter outer boot may lower manufacturing cost.

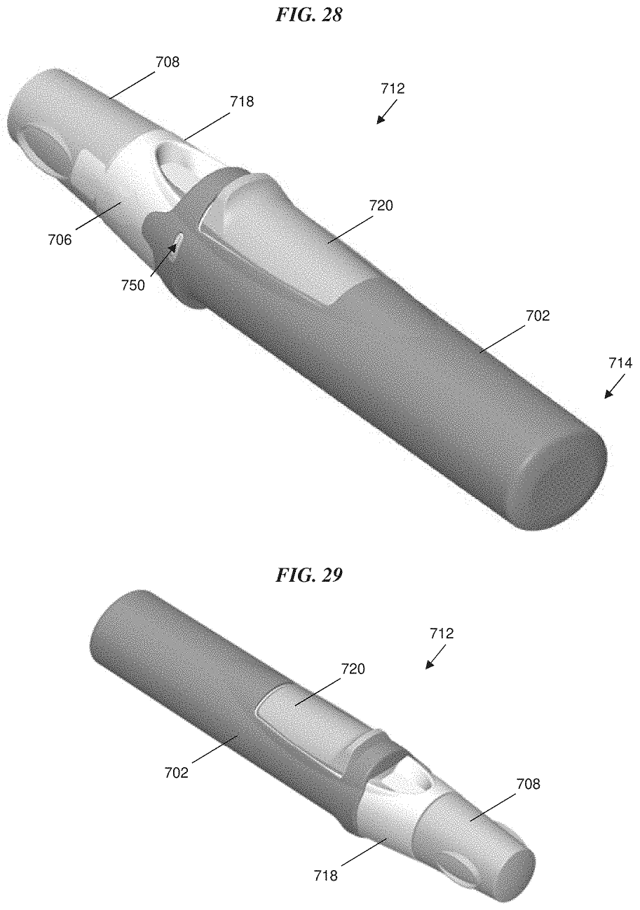

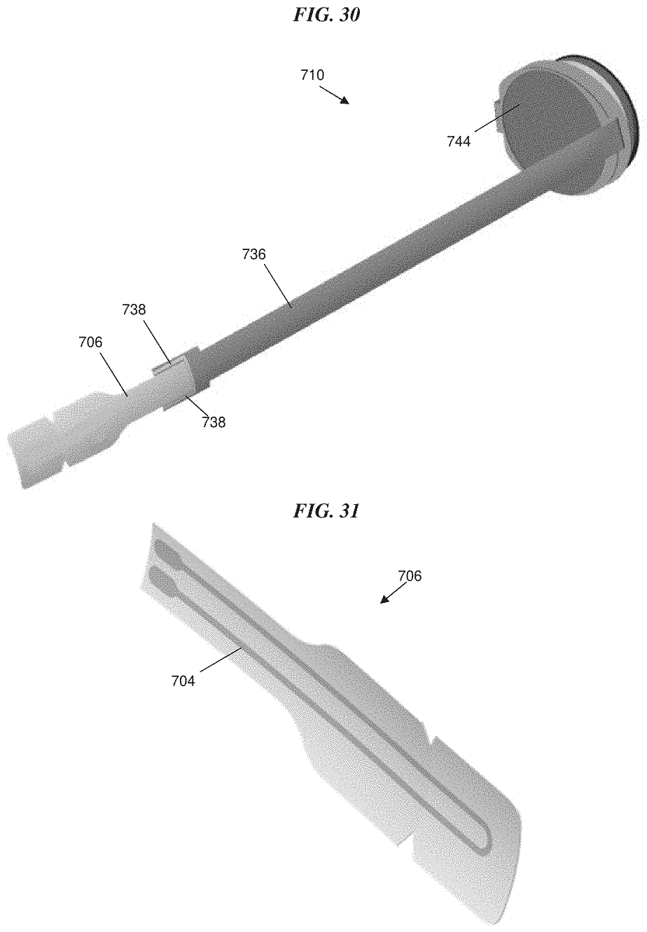

FIGS. 28 and 29 illustrate another embodiment of a drug delivery device 712 with another embodiment of a sensing module 710 (FIG. 30) attached thereto. The sensing module 710 is generally configured and used similar to the sensing module 10 of FIGS. 1 and 2. The drug delivery device 712 in this illustrated embodiment is a jet autoinjector configured and used similar to the jet injector discussed above with respect to FIGS. 17 and 18. The device 712 is configured to inject the drug from a container (obscured in FIGS. 28 and 29) in a housing 718 of the device 712 and out of a needle (not shown) in response to manual actuation by a user of a trigger 720 of the device 712. The drug delivery device 712 also includes a removable cap 708 configured to be removed from a remainder of the device 712 by a user to expose the needle. The device 712 also includes an outer boot 702 disposed over the sensing module 710 similar to the sensor module's disposal under the outer boot 402 of FIGS. 17 and 18, although as mentioned above the sensing module 710 can instead be on an outer surface of the outer boot 702 or be within the outer boot 702. The sensing module 710 is shown as a standalone element in FIG. 30 with another embodiment of a tab 706 coupled thereto. The tab 706 is shown as a standalone element in FIG. 31. The tab 706 is the same as the tab 406 of FIG. 15 except that the tab 706 has a different size and shape at least at its proximal end to interface properly with the sensing module 710, e.g., with a flexible circuit 736 thereof. Also, a conductive trace 704 is provided on the tab 706 using conductive tape in this illustrated embodiment.

The sensing module 710 is similar to the sensing module 410 of FIG. 15. The sensing module's electronic components are located at an end 714 of the drug delivery device 712 that is opposite to the end with the cap 708. However, unlike the device 412 of FIGS. 17 and 18, the device 512 of FIGS. 27 and 28, and the device 612 of FIGS. 26 and 27, the device end 714 is not protruding, e.g., does not have an enlarged diameter compared to the drug delivery device's housing. A protruding end may help indicate that a drug delivery device has a sensing module attached thereto. Not having a protruding end may allow a drug delivery device to have a more aesthetically appealing profile.

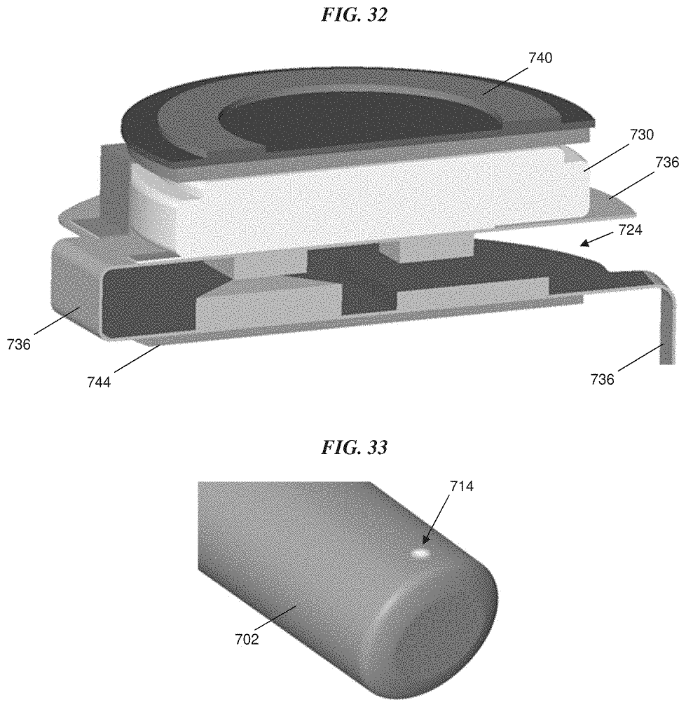

As shown in FIG. 32, the sensing module 710 in this illustrated embodiment has a “one-layer” configuration similar to that discussed above with respect to FIG. 23. The flexible circuit 736 extends from the tab 706 to have a portion positioned below the electronic components on the sensing module's PCB 724 and wraps around both the PCB 724 and the electronic components on the PCB 724 to have another portion positioned between the PCB 724 and the sensing module's power source 730. The power source 730 in this illustrated embodiment is a coin cell battery. The sensing module 710 also includes a receiver coil 740 similar to the receiver coil 440 of FIG. 19.

The sensing module 710 in this illustrated embodiment includes an LED on the PCB 724. As shown in FIG. 33, light emitted from the LED is configured to be visible through the outer boot 702. As mentioned above, the light can be used to indicate various conditions as programmed for the sensing module's processor, such as sensing status of the sensing module 710 (e.g., light on when the sensing module's sensor(s) are gathering data and light off when the sensor(s) are not gathering data), to show power status of the sensing module 710 (e.g., light on when the power source 730 is providing power, corresponding to the tab 706 being in its second position as having been removed, and light off when the power source 730 is not providing power, corresponding to the tab 706 being in its first position as being coupled to the sensing module 710).

As shown in FIG. 30, the flexible circuit 736 in this illustrated embodiment includes a guide marker 738 thereon. The guide marker 738 is configured to help guide placement of the tab 706 relative to the flexible circuit 736 during manufacturing to help ensure that the tab's conductive trace 704 is properly electronically coupled to the flexible circuit 736.

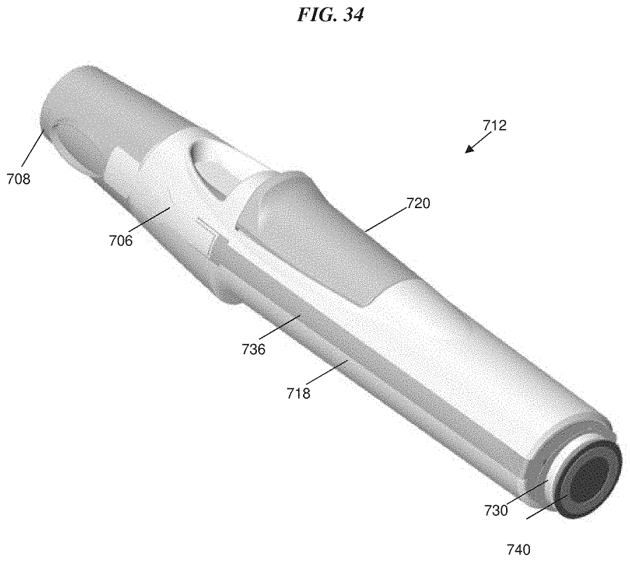

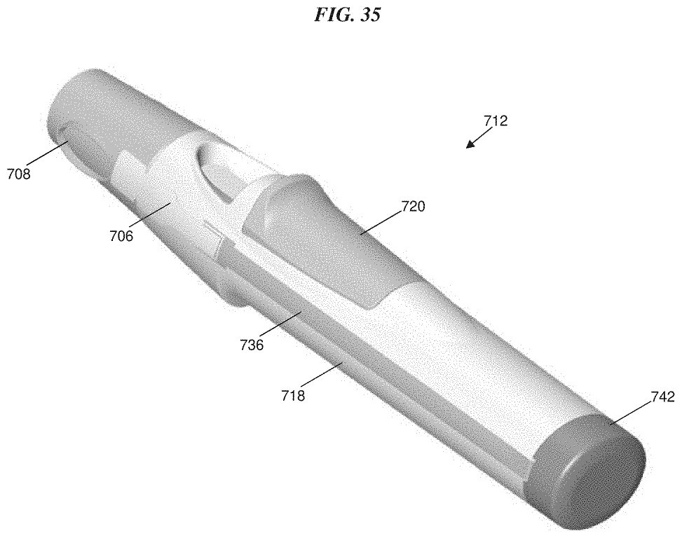

FIG. 34 shows the sensing module 710 and the tab 706 attached to the device 712 before an end cap 742 (FIG. 35) is attached to the device 712 to provide protection for the sensing module 710. A bottom of the sensing module 710 is attached to a top of the device 712, e.g., a top outer surface of the housing 718. To facilitate this attachment, the sensing module 710 includes an adhesive layer 744 on a bottom thereof, as shown in FIGS. 30 and 32. The adhesive layer 744 includes adhesive tape in this illustrated embodiment but can have other forms. The outer boot 702 is put into position over the device 712 as illustrated in FIG. 35 to result in the device 712 of FIGS. 28 and 29. The outer boot 702 can be configured to hold the end cap 742 in place, but in some embodiments, an adhesive and/or other attachment mechanism can be used to help hold the end cap 742 to the sensing module 710 before application of the outer boot 702 and/or an adhesive and/or other attachment mechanism can be used to help hold the end cap 742 to the outer boot 702 after the end cap's placement over the sensing module 710. In this illustrated embodiment the outer boot 702 has a window 750 (FIG. 28) formed therein as a hole to allow for visualization of the tab 706 therethrough to help ensure proper alignment of the tab 706 with respect to the flexible circuit 736.

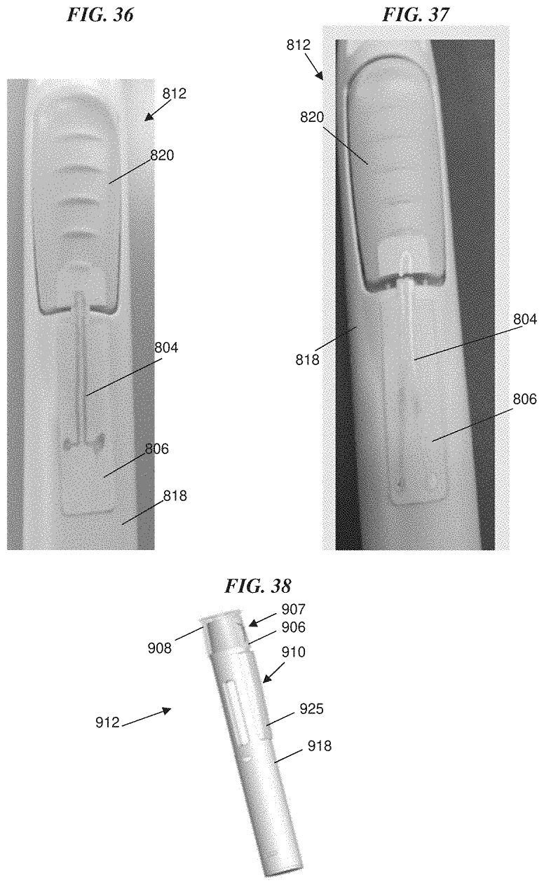

FIGS. 36 and 37 illustrate another embodiment of a drug delivery device 812 with another embodiment of a tab 806 attached thereto. The tab 806 includes a conductive trace 804 and is configured and used similar to other embodiments of tabs discussed above. The tab 806 is configured to couple to a sensing module, e.g., a flexible circuit thereof, similar to that discussed above with respect to other embodiments of tabs. The tab 806 is configured to move from a first position, in which the tab 806 is coupled to the sensing module (corresponding to a power source of the sensing module not providing power), to a second position, in which the tab 806 is torn or is not coupled to the sensing module (corresponding to the sensing module's power source providing power). With the tab 806 in the first position, which is shown in FIG. 36, the tab 806 prevents the flexible circuit from electrically connecting the power source with the sensing module's electronic components, which prevents the power source from providing power thereto. With the tab 806 in the second position, which is shown in FIG. 37, the flexible circuit electrically connects the power source with the electronic components of the sensing module, which allows the power source to provide power thereto. The tab 806 is thus configured to “wake up” the sensing module by moving from the first position to the second position. The sensing module's power source may therefore not run out of power before the end of drug delivery since power will not begin being used until the tab 806 is removed, e.g., the power source has zero shelf life power consumption.

In the illustrated embodiment of FIGS. 36 and 37, a first, lower portion of the tab 806 is attached to a trigger 820 of the drug delivery device 812, and a second, upper portion of the tab 806 is attached to a housing 818 of the drug delivery device 812. The conductive trace 804 is present on both of the first and second portions of the tab 806. When the trigger 820 is manually pressed by a user to cause drug delivery, the depression of the trigger causes the tab 806 to tear to separate the first and second portions of the tab 806 from one another and thereby cause a break in the conductive trace 804 and the tab 806 to move from the first position to the second position.

In some embodiments, the tab can include a sensor configured to gather motion data. The sensor configured to gather motion can include a communication interface configured to transmit data to an external source as discussed above and/or the sensor can be configured to transmit gathered data to a processor of the sensing module for communication to an external source via the sensing module's communication interface. For example, the tab 806 of FIGS. 36 and 37 can include a sensor configured to detect motion of the trigger 820, which may allow for detecting when drug delivery has begun, e.g., by detected motion of the trigger 820 being pressed, and/or for detecting when drug delivery has been completed, e.g., by detected motion of the trigger 820 being released after being pressed.

Whether or not the tab 806 includes a sensor configured to gather motion data, the tab 806 in some embodiments can include a magnet on the first portion thereof and a Hall effect sensor can be attached to the drug delivery device housing 818. The Hall effect sensor can thus be configured to detect movement of the trigger 820 since the magnet will move with the trigger 820 during depression of the trigger 820 and during release of the trigger 820. In some embodiments, instead of being attached to the tab 806, the magnet can be attached elsewhere with or without the tab 806 being used with the device 812. For example, a sensing module such as the sensing module 210 of FIG. 6 can include the Hall effect sensor and be attached to the housing 818.

FIG. 38 illustrates another embodiment of the drug delivery device 312 of FIGS. 10-12 with another embodiment of a sensing module 910 attached thereto. The sensing module 910 is attached non-removably to an outer surface of the device 312, although as mentioned above the sensing module 910 can instead be removably attached to the device 312. The outer surface is an outer surface of the drug delivery device's housing 318, but the sensing module 910 can be attachable to another outer surface of the drug delivery device 312 as discussed herein. The sensing module 910 is attached to the device 312 with an adhesive, e.g., a layer of an adhesive on a bottom portion of a housing 925 of the sensing module 910. However, as discussed herein, the sensing module 910 can be attached to a drug delivery device in other ways. The sensing module 910 can be attached anywhere on the housing 318 but in this illustrated embodiment is attached adjacent to the drug delivery device's cap 308 to facilitate use of a tab 906. The tab 906 is shown as a standalone element in FIG. 41. The tab 906 is the same as the tab 306 of FIG. 13 except that the tab 906 has a different size and shape at least at its proximal end to interface properly with the sensing module 910, as discussed further below. Also, a conductive trace 904 is provided on the tab 906 in this illustrated embodiment.

The sensing module 910 is generally configured and used similar to the sensing module 310 of FIGS. 10-13. In this illustrated embodiment, the housing 925 of the sensing module 910 is longer than the housing of the sensing module 310 and thus extends along more of the drug delivery device's longitudinal length than the housing of the sensing module 310. The longer housing 925 provides more space within the housing 925 for components of the sensing module 910. The sensing module 910 may thus have one or more enhanced features than a smaller sensing module, such as the sensing module 310 of FIGS. 10-13, such as greater processing capability (e.g., by having a larger processor and/or a greater number of processors for more processing capability than a smaller processor), more available memory storage (e.g., by having a larger memory and/or a greater number of memories for greater maximum storage than a smaller memory), more available power (e.g., by having a larger power source and/or a greater number of power sources for more available on-board power), greater communication capability (e.g., by a having a more robust communication interface and/or a greater number of communication interfaces for more range and/or for a greater number of available wireless techniques), etc.

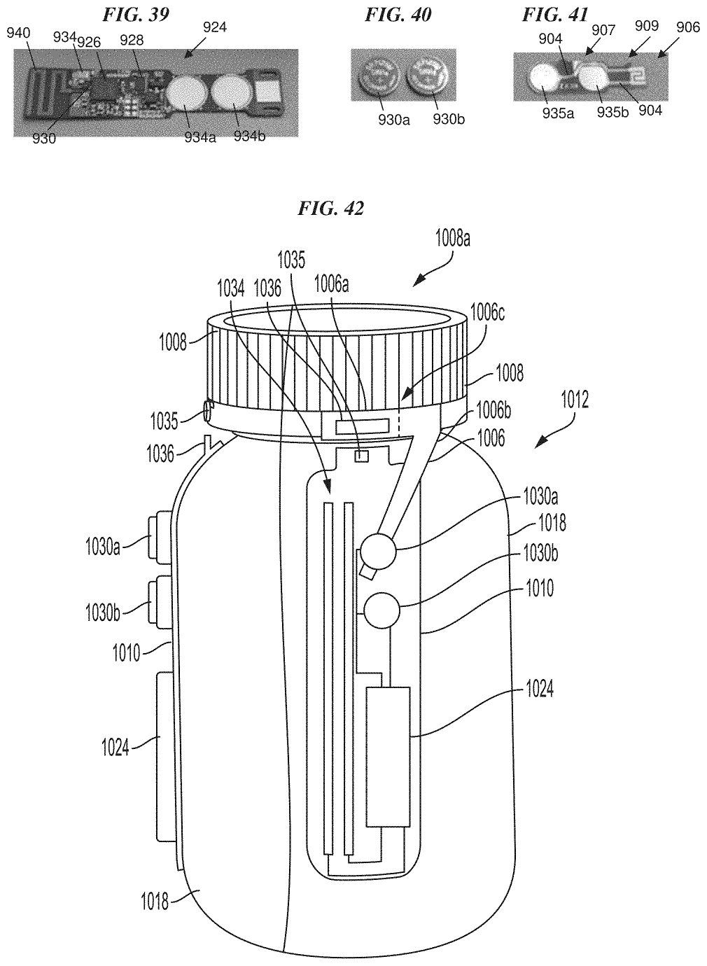

The sensing module 910 includes a variety of electronic components to facilitate the gathering of data and the transmission of gathered data to an external source similar to that discussed above regarding the sensing module 310 of FIGS. 10-13. As shown in FIGS. 39 and 40, the sensing module 910 includes a PCB 924 that includes a processor 926, a memory 928, a communication interface 930, a sensor 934, a receiver coil 940, and first and second contact pads 934a, 934b. The PCB 924 is disposed in the sensing module's housing 925 similar to that discussed above regarding the sensing module 310 of FIG. 13. The sensing module 910 also includes a power source in the form of first and second coin cell batteries 930a, 930b. The first and second power sources 930a, 930b are configured to operatively engage the first and second contact pads 934a, 934b, respectively, as discussed further below. The sensing module 910 including two power sources 930a, 903b may allow for the sensing module 910 to have more available on-board power than other sensing modules that include only one power source.

The power source 930a, 930b is configured to selectively provide power to one or more of the sensing module's electronic components, e.g., the processor 926, the communication interface 932, the sensor 934, etc., similar to that discussed above regarding the power source 330 of FIG. 13. The power source 930a, 930b is configured to not provide power when the tab 906 is coupled to the sensing module 910 and is configured to provide power when the tab 906 is torn or is not coupled to the sensing module 910. The tab 906 is shown coupled to the sensing module 910 in FIG. 38. The tab 906 is configured to move from a first position, in which the tab 906 is coupled to the sensing module 910 (corresponding to the power source 930a, 930b not providing power), to a second position, in which the tab 906 is torn or is not coupled to the sensing module 910 (corresponding to the power source 930a, 930b providing power). With the tab 906 in the first position, the tab 906 is located between the PCB 924 and the power source 930a, 930b and thereby prevents the first and second power sources 930a, 930b from contacting the first and second contact pads 934a, 934b, respectively. The power sources 930a, 930b are thus not electrically connected with the electronic components of the PCB 924 with the tab 906 in the first position since the tab 906 interrupts current flow. Instead of engaging the first and second contact pads 934a, 934b of the PCB 924, the first and second power sources 930a, 930b engage first and second contact pads 935a, 935b, respectively, of the tab 906 with the tab 906 in the first position. With the tab 906 in the second position, the first and second power sources 930a, 930b contact the first and second contact pads 934a, 934b, respectively, which allows the power source 930a, 930b to provide power to the electronic components of the PCB 424.

Similar to that discussed above regarding the tab 306 of FIG. 13, the tab 906 is configured to be removed from the sensing module 910, e.g., to move from the first position to the second position by sliding out of the sensing module's housing 925, in response to removal of the drug delivery device's cap 908 by a user. The tab 906 has a first, lower portion 907 located outside of the sensing module 910 and attached to the cap 408, as shown in FIG. 38, such as by being adhered thereto with adhesive and/or other attachment mechanism. The tab 906 has a second, upper portion 909 extending from the first portion 907 and extending into the sensing module's housing 925 and into contact with the first and second power sources 930a, 930b.

FIG. 42 illustrates another embodiment of a sensing module 1010 configured to gather data for one or more parameters related to a drug and to transmit data indicative of the gathered data to an external source configured to analyze the data received from the sensing module 1010. The sensing module 1010 is generally configured and used similar to the sensing module 10 of FIGS. 1 and 2. FIG. 42 shows the sensing module 1010 attached to a pill bottle 1012 configured to contain a drug therein in the form of pills. FIG. 42 shows a single sensing module 1010 in two views on the pill bottle 1012, a front view (sensing module 1010 on the right in FIG. 42) and a side view (sensing module 1010 on the left in FIG. 42).

The pill bottle 1012 in this illustrated embodiment is a standard pill bottle including a housing 1018 configured to contain the pills therein. The pill bottle 1012 also includes a removable cap 1008 configured to be removed from the housing 1018 by a user to access the pills in the housing 1018. The sensing module 1010 is attached to an outer surface of the housing 1018, which may facilitate retrofitting of the sensing module 1010 onto an existing pill bottle and/or may ease incorporation of the sensing module 1010 into a pill bottle's manufacturing process since the sensing module 1010 can be attached to the pill bottle's outer surface after the pill bottle has otherwise been filled with pills and closed with a removable cap.

The sensing module 1010 includes a variety of electronic components to facilitate the gathering of data and the transmission of gathered data to an external source similar to that discussed above regarding the sensing module 10 of FIGS. 1 and 2. The electronic components of the sensing module 1010 includes a PCB 1024, a power source in the form of first and second batteries 1030a, 1030b, a sensor 1034, and a reed switch 1036. The PCB 1024 includes various electronic components as discussed above, e.g., a processor, a memory, a communication interface, etc. The PCB 1024 and the sensor 1034 are flexible in this illustrated embodiment, which may facilitate smooth, close positioning of the sensing module 1010 on the pill bottle's curved outer surface. In other embodiments, however, the PCB 1024 and/or the sensor 1034 can be rigid.

The sensor 1034 can include any of a variety of sensors, as discussed herein. In an exemplary embodiment, the sensor 1034 includes a level sensor, e.g., a capacitive-based liquid level sensor such as the TIDA-00317 capacitive-based liquid level sensor available from Texas Instruments Incorporated of Dallas, Tex.

The power source 1030a, 1030b is configured to selectively provide power to one or more of the sensing module's electronic components, similar to that discussed above regarding the power source 330 of FIG. 13. The power source 1030a, 1030b is configured to not provide power when a tab 1006 is coupled to the sensing module 1010 and is configured to provide power when the tab 1006 is torn or is not coupled to the sensing module 1010. The tab 1006 is shown coupled to the sensing module 1010 in FIG. 42.

The tab 1006 includes a first portion 1006a and a second portion 1006b that is configured to be separated from the first portion 1006a at a tear line 1006c. The first portion 1006a of the tab 1006 is attached to the cap 1008 and is aligned with a magnet 1035. The magnet 1035 can have a variety of configurations. For example, the magnet 1035 can be printed, e.g., ink jet printed with magnetic ink, on the tab 1006, e.g., on the first portion 1006a thereof. For another example, the magnet 1035 can be put on a heat shrink wrap around the tab 1006, e.g., the first portion 1006a thereof. For yet another example, the magnet 1035 can be attached to the tab 1006 by being adhered thereto with an adhesive. For still another example, the magnet 1035 can be attached to or printed on the cap 1008 with the first portion 1006a of the tab 1006 then being positioned to overlie the magnet 1035.

The tab 1006 is configured to move from a first position, in which the tab 1006 is coupled to the sensing module 1010 (corresponding to the power source 1030a, 1030b not providing power), to a second position, in which the tab 1006 is torn or is not coupled to the sensing module 1010 (corresponding to the power source 1030a, 1030b providing power). The first portion 1006a of the tab 1006 is operatively engaged with the power source, e.g., the first power source 1030a, with the tab 1006 in the first position. The magnet 1035 is aligned with the reed switch 1036 with the tab 1006 in the first position.

Similar to that discussed above regarding the tab 306 of FIG. 13, the tab 1006 is configured to be removed from the sensing module 1010, e.g., to move from the first position to the second position, in response to movement of the cap 1008 by a user. The cap 1008 is configured to be rotated counter-clockwise relative to the housing 1018, as shown by arrow 1008a. When the cap 1008 is rotated counter-clockwise relative to the housing 1018 (and to the sensing module 1010 attached to the housing 1018), the tab 1006 will be pulled out of engagement from the power source, e.g., the second portion 1006b of the tab 1006 will move out of engagement form the first power source 1030a, and the magnet 1035 will rotate with the cap 1008 and become misaligned from the reed switch 1036. The reed switch 1036 will therefore detect a change in magnetic field. The change in magnetic field is indicative of the cap 1008 being removed from the housing 1018. The reed switch 1036 is configured to communicate the detected change to the PCB 1024, e.g., to a processor thereof, so as to inform the PCB 1024 of cap 1008 removal. Cap 1008 removal is indicative of pill(s) being removed from the pill bottle 1002 and taken by a patient in accordance with drug administration instructions.

The tab 1006 is only removed from the sensing module 1010 the first time the cap 1008 is removed from the housing 1018. Thus, when the cap 1008 is removed from the housing 1018 for the first time, the second portion 1006b of the tab 1006 will be dangling from the cap 1008 and can be removed by tearing the tab 1006 at the tear line 1006c. The second portion 1006b of the tab 1006 will therefore not be in a user's way during subsequent use of the pill bottle 1012.

When the cap 1008 is reattached to the housing 1018, the magnet 1035 and the reed switch 1036 will again be aligned. The reed switch 1036 will therefore detect a change in magnetic field. The change in magnetic field is indicative of the cap 1008 being reattached to the housing 1018. The reed switch 1036 is configured to communicate the detected change to the PCB 1024, e.g., to a processor thereof, so as to inform the PCB 1024 of cap 1008 reattachment. Cap 1008 removal and cap 1008 reattachment can occur any number of subsequent times, with the reed switch 1036 detecting magnetic field changes and communicating the detected changes to the PCB 1024 so as to repeatedly indicate pill(s) being removed from the pill bottle 1002 and taken by a patient.

In other embodiments, the cap 1008 can be configured to be removed from the housing 1018 by being rotated clockwise relative to the housing 1018 (and the sensing module 1010 attached to the housing 1018), with the tab 1006 and power source arranged accordingly to operate as discussed above.

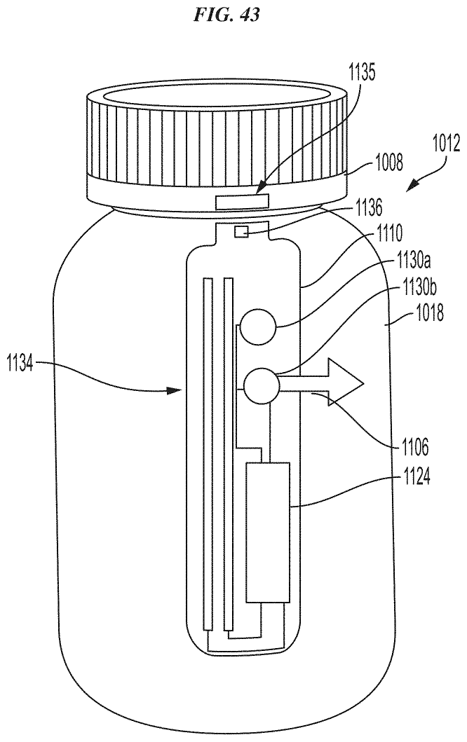

FIG. 43 illustrates another embodiment of a sensing module 1110 configured to gather data for one or more parameters related to a drug and to transmit data indicative of the gathered data to an external source configured to analyze the data received from the sensing module 1110. FIG. 43 shows the sensing module 1110 attached to the pill bottle 1012 of FIG. 42 but can be similarly used with other pill bottles. The sensing module 1110 is generally configured and used similar to the sensing module 1010 of FIG. 42, e.g., includes a PCB 1124, a power source in the form of first and second batteries 1130a, 1130b, a sensor 1134, and a reed switch 1136.

A tab 1106 is configured to move from a first position, in which the tab 1106 is coupled to the sensing module 1110 (corresponding to the power source 1130a, 1130b not providing power), to a second position, in which the tab 1106 is torn or is not coupled to the sensing module 1110 (corresponding to the power source 1130a, 1130b providing power). In the illustrated embodiment of FIG. 42, removing the cap 1008 from the housing 1018 is configured to automatically release the tab 1006 from the sensing module 1010, e.g., from the power source, and to misalign the magnet 1035 and the reed switch 1036. In the illustrated embodiment of FIG. 43, removing the cap 1008 is configured to similarly misalign a magnet 1135 and the reed switch 1136, but the tab 1106 is not automatically released from the sensing module 1110. Instead, in the illustrated embodiment of FIG. 43, the tab 1106 is configured to be manually removed from the sensing module 1110 by being pulled by a user. As in this illustrated embodiment, the tab 1106 can be shaped like an arrow to indicate a direction in which the tab 1106 should be pulled to be removed from the sensing module 1110. An arrow can additionally or alternatively be printed on the tab 1006. The tab 1106 can have other shapes other than arrow-shaped, such as rectangular, triangular, hourglass-shaped, pear-shaped, I-shaped, etc.

A user of the pill bottle 1012 can be provided with instructions to remove the tab 1106 before the cap 1008 is first removed from the housing 1018. In this way, the electronic components of the sensing module 1100 can be “woken up” before the cap 1008 is first removed from the housing 1018. The tab 1106 can be pulled by a patient who will take the pills in the pill bottle 1012. Alternatively, the tab 1106 can be pulled by an authorized user such as a health care provider, a pharmacist, or other authorized user who provides the pill bottle 1012 to a patient who will take the pills in the pill bottle 1012. The tab 1106 being pulled by an authorized user instead of the patient may help ensure that the tab 1106 is pulled and the sensing module 1100 “wakes up” before the cap 1108 is removed for the first time from the housing 1018.

The embodiments of FIGS. 42 and 43 use a magnet and a reed switch in detecting cap removal, but other implementations are possible. For example, a magnet can be attached to a pill bottle cap similar to the magnets 1035, 1135 discussed above, and a sensing module similar to the sensing modules 1010, 1110 discussed above can include a Hall effect sensor configured to detect a change in magnetic field similar to the reed switches 1036, 1136 discussed above. For another example, a sensing module similar to the sensing modules 1010, 1110 discussed above can include an infrared (IR) transmitter and receiver configured to emit an IR light toward a removable cap of a pill bottle to which the sensing module is attached. The cap can either be reflective or include a reflective area toward which the IR light is emitted. The reflective cap or the reflective area of the cap is configured to reflect the IR light to the IR receiver. The cap being removed from the pill bottle's housing will thus interrupt the IR light reflection and receipt, thereby indicating that the cap has been removed.

The manually pullable tab 1106 of FIG. 43 is the only tab used with the sensing module 1110 and the pill bottle 1012. The automatically pullable tab 1006 of FIG. 42 is the only tab used with the sensing module 1110 and the pill bottle 1012. The embodiments of sensing modules used with tabs discussed above with respect to FIGS. 10-13 (sensing module 310 and tab 306), FIGS. 15 and 16 (sensing module 410 and tab 406), FIGS. 24 and 25 (sensing module 410 and tab 506), FIGS. 26 and 27 (sensing module 410 and tab 606), and FIGS. 28 and 29 (sensing module 710 and tab 706) involve use of a single tab with a drug delivery device. The tab 806 of FIGS. 36 and 37 is the only tab used with the drug delivery device 812.

In other embodiments, a drug delivery device or pill bottle can be used with two tabs. A first one of the tabs can be operatively coupled to a sensing module and configured to be manually moved to “wake up” the sensing module, similar to the manually pullable tab 1106 of FIG. 43. The sensing module can thus begin collecting data using one or more sensors of the sensing module, such as date, time, temperature, humidity, etc., before drug delivery begins. The sensing module can thus gather data for a period of time before drug delivery begins that may indicate that the drug was exposed to adverse conditions, e.g., too high a temperature, too low a temperature, too high humidity, too high pressure, etc., before delivery and may thus not perform as expected. The sensing module can be configured to gather data continuously after the sensing module “wakes up.” Alternatively, the sensing module can be configured to gather data on a predetermined periodic basis after the sensing module “wakes up,” such as once every minute, once every thirty minutes, once every hour, once every two hours, once every twenty-four hours, etc. In embodiments in which the sensing module is configured to monitor more than one parameter, each parameter can be monitored on the same time schedule, e.g., each being sensed once every minute, once every thirty minutes, once every hour, once every two hours, once every twenty-four hours, etc., or each can be monitored on its own schedule that is different from at least one other of the monitored parameters.

A user of the drug delivery device can be provided with instructions to remove the first tab a certain amount of time before expected drug delivery, e.g., forty-eight hours before expected drug delivery, twenty-four hours before drug delivery, at least forty-eight hours before drug delivery, at least twenty-four hours before drug delivery, one hour before drug delivery, at least one hour before drug delivery, etc. Removing the first tab a certain amount of time before expected drug delivery may help ensure that the sensing module's power source does not run out of power before the end of drug delivery (or before a pill supply is exhausted) since power will not begin being used until the user removes the first tab. Removing the first tab a certain amount of time before expected drug delivery may facilitate data analysis by providing more data for comparison purposes, e.g., more spatial orientation data to determine a drug delivery device's movements, more temperature data to determine if the drug experienced temperature swings before delivery etc.

A second one of the tabs can be configured to be automatically moved in response to a user action, e.g., cap removal, trigger actuation, etc., that occurs at a time the drug delivery process begins or shortly before the drug delivery process begins from a drug delivery device or shortly before pill(s) are removed from a pill bottle. Removing the second tab fully “wakes up” the drug delivery device or pill bottle so the power source is providing power to electronic components to gather data and allow for drug delivery or pill access as appropriate for the particular drug delivery device or pill bottle. With the drug delivery device or pill bottle fully “awake,” all electronic functionality of the drug delivery device or pill bottle is available. Examples of such second tabs include tabs similar to the tabs discussed above that are each configured to move in response to a user action in the form of cap removal. Examples of electronic components of a drug delivery device that can begin receiving power in response to the second tab being removed include components configured to gather data regarding the drug delivery process, e.g., an accelerometer, a microphone, a proximity sensor, etc.

The positions of the first and second tabs can define power modes of the drug delivery device or pill bottle. Before the first and second tabs are removed, the drug delivery device or pill bottle can be in a no power mode because the power source is not yet providing power to electronic components. After the first tab is removed and before the second tab is removed, the drug delivery device or pill bottle can be in a low power mode in which the power source is providing power to electronic components to gather data before drug delivery begins or before pills are removed from the pill bottle. After the first and second tabs are moved, the drug delivery device or pill bottle can be in a high power mode in which the power source is providing power to electronic components to gather data and allow for drug delivery or pill access as appropriate for the particular drug delivery device or pill bottle. Less power is required from the power source for data gathering than for the data gathering in addition to allowing for drug delivery or pill access, so the low power mode may help conserve power and thereby help ensure that the power source has sufficient power throughout drug delivery or pill access in the high power mode. Less power may also be required for data gathering in the low power mode since less data may be gathered before drug delivery or pill access begins than after drug delivery or pill access begins, so the low power mode may help conserve power and thereby help ensure that the power source has sufficient power throughout drug delivery or pill access in the high power mode.

In some embodiments, instead of the first tab being configured to be manually moved by a user to “wake up” the sensing module, the first tab can be configured to be automatically moved in response to a user action to “wake up” the sensing module. The user action to “wake up” the sensing module is different from the user action that moves the second tab. The user action configured to “wake up” the sensing module can include opening a package containing the drug administration device (or pill bottle) therein. The first tab can be operatively connected to each of the package and the drug administration device (or pill bottle) such that opening of the package causes the first tab to be removed from the drug administration device (or pill bottle). For example, the first tab can be connected to a blister pack lid that is pulled off by a user to gain access to the drug administration device (or pill bottle) in the blister pack. The pulling off of the lid can automatically cause the first tab to be removed from the sensing module. For another example, the first tab can be connected to a portion of a cardboard box package, e.g., a side thereof marked as the side of the package to open, such that moving that portion of the box to gain access to the drug administration device (or pill bottle) in the box automatically cause the first tab to be removed from the sensing module.

A drug delivery device with a sensing module and first and second tabs attached thereto can have a variety of configurations. In one exemplary embodiment, the first tab and the second tab can each be a tab that acts as an insulator such that an open circuit exists to prevent the drug delivery device's power source from providing power to electronic components of the sensing module as discussed above. Examples of such tabs include the tab 306 of FIGS. 10-13, the tab 906 of FIG. 38, and the tab 1106 of FIG. 43. The power source can include a first power source operatively coupled with the first tab (with the first tab in its first position) and a second power source operatively coupled with the second tab (with the second tab in its first position). Instead of the first tab being configured to be automatically removed from the sensing module in response to removal of the drug delivery device's cap, such as with the tab 306 of FIGS. 10-13 and the tab 906 of FIG. 38, the first tab is configured to be manually removed from the sensing module similar to that discussed above regarding the manually pullable tab 1106 of FIG. 43. The removal of the first tab “wakes up” the sensing module as discussed above, e.g., moves the drug delivery device from a no power mode to a low power mode. Additionally, unlike automatically movable insulator tabs such as the tab 306 of FIGS. 10-13 that have a first, lower portion fixed to the drug delivery device, a first, lower portion of the first tab that is located outside of the sensing module is not fixed to the cap or to another portion of the drug delivery device in order to facilitate manual grasping and removal of the first tab. The first tab's removal from the sensing module can be configured to allow the first power source to begin providing power to a first one or more electronic components of the sensing module to which the first power source is operatively coupled. Examples of the first one or more of the electronic components include sensors configured to gather data. The second tab can be configured to be automatically removed from the sensing module in response to removal of the drug delivery device's cap, such as with the tab 306 of FIGS. 10-13 and the tab 906 of FIG. 38. The movement of the second tab from its first position to its second position, e.g., by removing a cap of the drug delivery device, etc., fully “wakes up” the drug delivery device as discussed above, e.g., moves the drug delivery device from the low power mode to a high power mode. The second tab's removal from the sensing module can be configured to allow the second power source to begin providing power to a second, different one or more of electronic components of the sensing module to which the second power source is operatively coupled. Examples of the second one or more of the electronic components include components configured to gather data regarding the drug delivery process, e.g., an accelerometer, a microphone, a proximity sensor, etc.

In another exemplary embodiment, the first tab can be a tab that acts as an insulator such that an open circuit exists to prevent the drug delivery device's power source from providing power to electronic components of the sensing module as discussed above. Examples of such tabs include the tab 306 of FIGS. 10-13, the tab 906 of FIG. 38, and the tab 1106 of FIG. 43. Instead of the first tab being configured to be automatically removed from the sensing module in response to removal of the drug delivery device's cap, such as with the tab 306 of FIGS. 10-13 and the tab 906 of FIG. 38, the first tab is configured to be manually removed from the sensing module similar to that discussed above regarding the manually pullable tab 1106 of FIG. 43. The removal of the first tab “wakes up” the sensing module as discussed above, e.g., moves the drug delivery device from a no power mode to a low power mode. Additionally, unlike automatically movable insulator tabs such as the tab 306 of FIGS. 10-13 that have a first, lower portion fixed to the drug delivery device, a first, lower portion of the first tab that is located outside of the sensing module is not fixed to the cap or to another portion of the drug delivery device in order to facilitate manual grasping and removal of the first tab. The second tab can be a tab that includes a conductive trace thereon, where the second tab is configured to interrupt power from the power source to electronic components of the drug delivery device until the conductive trace is torn or de-coupled from the sensing module. Examples of such tabs include the tab 406 of FIGS. 15-17 configured to move from the first position to the second position in response to cap 408 removal, the tab 506 of FIG. 24 configured to move from the first position to the second position in response to cap 508 removal, the tab 606 of FIG. 26 configured to move from the first position to the second position in response to cap 608 removal, the tab 706 of FIG. 28 configured to move from the first position to the second position in response to cap 708 removal, and the tab 806 of FIGS. 36 and 37 configured to move from the first position to the second position in response to pressing of the trigger 820. The movement of the second tab from its first position to its second position, e.g., by removing a cap of the drug delivery device, by pressing a trigger of the drug delivery device, etc., fully “wakes up” the drug delivery device as discussed above, e.g., moves the drug delivery device from the low power mode to a high power mode.

The sensing modules discussed above are discussed with respect to non-training drug delivery devices but can each be similarly used with a drug delivery training device configured to simulate delivery of a drug for training purposes. The drug delivery training devices are configured and used similar to the drug delivery devices discussed above but have one or more features present to prevent actual drug delivery, such as by no drug or other liquid being contained therein, by the drug delivery training device not including a needle, or by saline or other safe non-drug being delivered instead of a drug. The drug delivery training devices are also configured, as will be appreciated by a person skilled in the art, to be reset after each use to allow the drug delivery training device to be re-used.

A sensing module used with a drug delivery training device can allow for data gathered during a drug delivery process to be used in real time with the simulated drug delivery process to assist in training the user during use of the training device and/or can be used after the simulated drug delivery process to help the user understand success/failure of the process and have a more helpful and/or faster training experience. Data gathered with respect to the drug delivery training device can also be used when the user uses an actual drug delivery device to help ensure that the user maintains good practices developed during training.

As will be appreciated by a person skilled in the art, a drug delivery training device can be used in cooperation with an application (also referred to herein as an “app”) installed on a computer system accessible by the trainee. Data gathered by the sensing module can be communicated to the computer system (as the external source that is located external to the drug delivery training device) using the sensing module's communication interface. The computer system can be configured to provide data as discussed herein to the user via the app after the simulated drug delivery process to help the user understand success/failure of the process. Alternatively or in addition, the computer system can be configured to provide data as discussed herein to the user via the app in real time with the simulated drug delivery process. An app for a drug delivery training device can, as will be appreciated by a person skilled in the art, walk the user through the process as part of the training. The data gathered by the sensing module and communicated to the computer system can allow the app to provide real time feedback to the user about potentially detected problems with the drug delivery process. Examples of such problems include an improper angle (spatial orientation) of the device during the process, a trigger or a plunger not being sufficiently depressed, the device's cap not being removed when the device is in position for simulated drug delivery, the device's cap not being put back on after drug delivery, and the device being removed from the patient's skin before completion of the simulated drug delivery. The app can be “smart” in that the app can be configured to learn mistake(s) the user is making when practicing with the drug delivery training device and can be configured to guide the user to correct the mistake(s) during subsequent practice with the drug delivery training device, e.g., by providing an instruction to help prevent the mistake(s) from occurring (e.g., to hold an injector perpendicular to the skin, to fully depress the device's trigger, etc.) or later during the user's use of an actual drug delivery device. As mentioned above, the app can similarly be used in connection with an actual drug delivery device to allow the app to provide real time feedback to the user about potentially detected problems with the drug delivery process and to learn mistake(s) that occur during drug delivery device use.

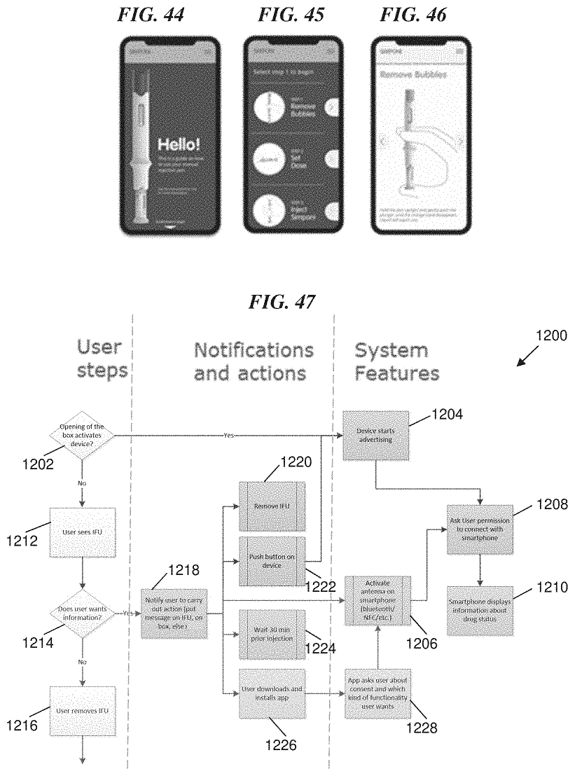

FIGS. 44-46 illustrate embodiments of app pages on one embodiment of a computer system (a mobile phone). FIG. 44 illustrates an embodiment of a welcome page showing a greeting and an image of the device that the user should be using for training. FIG. 45 illustrates an embodiment of a process page with step-by-step instructions of the drug delivery simulation process. Each step, e.g., priming (removing bubbles), setting dose, injecting the drug, etc. is selectable by the user in order to provide further information on how to successfully perform that step. FIG. 46 illustrates an embodiment of a priming step's page.

In an exemplary embodiment, data from the sensing module is incorporated into the pages for the steps. For example, if the sensed data indicates that the device is at an improper angle for priming or for injection (or for simulated injection in the case of a drug delivery training device) and/or that the device's removable cap has not been removed, a warning can appear on the priming page that a possible error has been detected. Information on how to correct the error can also be provided, e.g., an instruction of how to properly angle the device, an instruction to remove the cap, etc. For another example, if the sensed data indicates that the device's trigger has not been pushed, an instruction can appear on the injection page until the sensed data indicates that the device's trigger has been pushed. For still another example, if the sensed data indicates that the device's needle shield has not been moved to a position indicative of the device's needle having been fully exposed, an instruction can appear on the injection page until the sensed data indicates that the needle shield has moved a sufficient amount. For yet another example, if the sensed data indicates that the device is removed from the patient's skin before enough time has passed for injection to be completed (or for simulated injection to be completed in the case of a drug delivery training device), an error message can appear on the injection page indicating to the user that the device may have been removed prematurely from the patient. For another example, if the sensed data indicates that a step is performed out of sequence, an error message can appear on the current page indicating that a step was missed, such as if a plunger or trigger appears to be being pressed before a prior required step was completed. For yet another example, previously gathered sensed data (gathered during training or during actual device use) can be used to provide a message intended to correct previously detected mistake(s) whether the app is being used in training or in actual drug delivery, such as a message indicating the proper perpendicular angle relative to skin for proper drug injection, a message indicating that the device's trigger should be fully depressed to ensure drug delivery, a message indicating a duration of time the device should be held against skin during drug delivery, etc. For another example, the step-by-step instructions can begin with a list of one or more suggested remediations to address problem(s) identified by analyzing previously gathered sensed data, which may highlight the remediations to the user at the outset and thereby help the user remember to perform all steps correctly.

Similar to that discussed above regarding a drug delivery training device, the app can be used in connection with an actual drug delivery device to allow the app to walk the user through the drug delivery process and/or to provide real time feedback to the user about potentially detected problems with the drug delivery process. The app can also be configured to learn mistake(s) that occur during drug delivery device use similar to that discussed above. In embodiments in which the drug delivery device is used with an app, the external source to which the sensing module's communication interface communicates data can be the computer system providing the app. The computer system providing the app can be configured to communicate data received from the sensing module to a second external source such as a computer system located remotely from the sensing module, such as the central computer system 100 of FIG. 4. Alternatively or in addition, the sensing module can be configured to communicate data to the second external source.

FIG. 47 illustrates one embodiment of a method 1200 of a sensing module establishing communication with an external source, e.g., a mobile phone or other computer system, configured to run an app for use in conjunction with a drug delivery device. The method 1200 is described with respect to an actual drug delivery device but can be similarly used with a drug delivery training device or a pill bottle.

As mentioned above, in some embodiments, opening a package in which a drug delivery device is contained can cause a sensing module attached to the drug delivery device to “wake up.” “Waking up” the sensing module is also referred to herein as “activating” the sensing module. In the method 1200, if a user opening 1202 a package in which the drug delivery device is contained causes the sensing module to be activated, the sensing module's communication interface “wakes up” and starts 1204 advertising its presence, e.g., begins emitting a wireless signal. The external source includes a communication interface configured to receive the advertising signal, e.g., received by an antenna of the communication interface. The external source is a smartphone in this illustrated embodiment but can, as discussed herein, be another type of computer system. The antenna is activated 1206 in accordance with the external source's operation, such as by the external source being turned on, the external source's wireless capability being turned on, etc. The activation 1206 of the antenna can be before or after the sensing module starts 1204 advertising. In response to receiving the advertising signal from the sensing module, the external source asks 1208 a user for permission to connect the external source to the sensing module, which may be identified in the ask 1208 as the drug delivery device. The ask 1208 can be, for example, a prompt shown on a display of the external source. After receiving an affirmative response to the ask 1208 allowing the external source to connect to the sensing module, the external source displays 1210 information, e.g., via the app, about drug status as communicated to the external source from the sensing module. Drug status information can include, for example, when a next dose of drug is due for delivery, a type of the drug, drug expiration date, etc. The external source can additionally or alternatively display other information, such as information regarding the drug delivery device.

In the method 1200, if the user opening 1202 the package in which the drug delivery device is contained does not cause the sensing module to be activated, the user sees 1212 printed Instructions For Use (IFU) for the drug delivery device in the package. If the user decides 1214 that further information is not desired from the IFU, the user removes 1216 the IFU from the package (and/or from the drug delivery device) and can proceed to use the drug delivery device. The user may decide 1214 that further information is not desired for any of a variety of reasons, such as the user already being familiar with how to use the drug delivery device, the user not having access to an external source with which the sensing module could communicate, etc. The IFU can be attached to the drug delivery device to help ensure that the user sees 1212 the IFU and decides 1214 whether or not more information is desired before using the drug delivery device.

If the user decides 1214 that further information is desired from the IFU, the user is notified 1218 to take various actions. The notification 1218 can be provided to the user in one or more ways. For example, the IFU can provide the notification 1218 via written instruction. For another example, the notification 1218 can be provided on the package via written instruction. For yet another example, the notification 1218 can be provided on the drug delivery device, such as a written instruction printed on the drug delivery device and/or on a label, sticker, etc. on the drug delivery device.

In this illustrated embodiment, the notification 1218 includes four instructions, but more than four instructions or fewer than four instructions can be provided in other embodiments. One of the instructions instructs the user to remove 1220 the IFU from the package (and/or from the drug delivery device). Another one of the instructions instructs the user to provide 1222 an input to the drug delivery device and/or the sensing module attached to the drug delivery device. The input in this illustrated embodiment is a push of a button but can be another input, such as toggling of a switch, rotation of a knob, etc. The button (or switch, knob, etc.) is operatively connected to the communication interface of the sensing module. The input causes the communication interface of the sensing module to start 1204 advertising its presence, with the method 1200 continuing from the start 1204 of the advertising as discussed above. Another one of the instructions instructs the user to wait 1224 a certain amount of time before beginning drug delivery from the drug administration device. The certain amount of time is thirty minutes in this illustrated embodiment but can be another amount of time. The amount of time can be different for any of a variety of reasons, such as the type of drug, whether the drug must be stored in a refrigerator and be warmed to room temperature before delivery, whether the drug must be delivered within a particular amount of time relative to another drug being administered, etc. In some embodiments the user need not wait 1224 any time at all before beginning drug delivery from the drug administration device, in which case this instruction need not be provided. Another one of the instructions instructs the user to download and install 1226 the app on the user's smartphone (or other computer system) if the app is not already so installed on the user's smartphone (or other computer system). Once installed 1226 on the smartphone (or other computer system), the app prompts 1228 the user for consent, e.g., to accept the app's terms of use, to acknowledge the app's privacy terms, etc., and for the user to select desired functions of the app. In some embodiments, the user does not have a choice to select desired functionality of the app with the app instead having preset functionality. For example, the user can choose whether or not to receive audio instructions in addition to or instead of written instructions provided on a display of the smartphone (or other computer system). For another example, the user can choose a default language (English, Spanish, French, etc.). After the prompts have been fulfilled, the antenna is activated 1206 and the method 1200 continues as discussed above. In some embodiments, the antenna can be activated 1206 before any of the prompts have been fulfilled or in response to a particular prompt being fulfilled, such as the antenna being activated 1206 in response to the user providing consent.

As discussed herein, one or more aspects or features of the subject matter described herein, for example components of the central computer system 100, processor 24, power source 32, memory 28, communication interface 30, sensor 26, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communication network, e.g., the Internet, a wireless wide area network, a local area network, a wide area network, a wired network, a cellular network, etc. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example a user interface of the central computer system 100, can be implemented on a computer having a display screen, such as for example a cathode ray tube (CRT) or a liquid crystal display (LCD) or a light emitting diode (LED) monitor for displaying information to the user. The display screen can allow input thereto directly (e.g., as a touch screen) or indirectly (e.g., via an input device such as a keypad or voice recognition hardware and software).

The present disclosure has been described above by way of example only within the context of the overall disclosure provided herein. It will be appreciated that modifications within the spirit and scope of the claims may be made without departing from the overall scope of the present disclosure.

1. A sensing module for a drug delivery device, comprising:
a base configured to be attached to an outer surface of a drug delivery device;
a sensor located on the base and configured to gather data regarding at least one of date, time, vibration, temperature, sound, motion, humidity, pressure, fluid level, force, location, proximity, and spatial orientation;
a communication interface located on the base and configured to wirelessly transmit data to an external source; and
a processor located on the base and configured to receive data from the sensor indicative of the gathered data and to cause the communication interface to wirelessly transmit data indicative of the received data to the external source.

2. The sensing module of claim 1, further comprising a flexible circuit board with the sensor, the processor, and the communication interface thereon.

3. The sensing module of claim 1, further comprising a rigid circuit board with the sensor, the processor, and the communication interface thereon.

4. The sensing module of claim 1, wherein the base includes a housing with the sensor, the processor, and the communication interface disposed therein.

5. The sensing module of claim 4, further comprising a circuit board with the sensor, the processor, and the communication interface thereon, the circuit board being disposed within the housing.

6. The sensing module of claim 1, wherein the base includes a thin-film device, and the sensing module further comprises an adhesive configured to attach the thin-film device to the outer surface of the drug delivery device.

7. The sensing module of claim 1, wherein the base is configured to be non-removably attached to the outer surface of the drug delivery device.

8. The sensing module of claim 1, further comprising a power source configured to provide power to at least one of the sensor, the processor, and the communication interface.

9. The sensing module of claim 8, further comprising an insulator attached to the base in a first position, in which the insulator prevents the power source from providing the power to at least one of the sensor, the processor, and the communication interface;
wherein the insulator is configured to be manually moved by a user from the first position to a second position, in which the insulator allows the power source to provide the power to at least one of the sensor, the processor, and the communication interface.

10. The sensing module of claim 9, wherein the insulator includes a tab configured to be manually torn to move from the first position to the second position.

11. The sensing module of claim 9, further comprising a switch operatively connected to the power source;
wherein with the insulator in the first position the switch is in an open position, and with the insulator in the second position the switch is in a closed position.

12. The sensing module of claim 9, wherein the insulator includes a first tab;
the sensing module further comprises a second tab attached to the base in a third position, in which the sensor is not gathering the data;
the second tab is configured to be manually moved by a user from the third position to a fourth position; and
the movement of the second tab from the third position to the fourth position allows the sensor to begin gathering the data.

13. The sensing module of claim 8, further comprising a conductive trace configured to be manually moved by a user from a first position, in which the conductive trace prevents the power source from providing the power to at least one of the sensor, the processor, and the communication interface to a second position, in which the conductive trace allows the power source to provide the power to at least one of the sensor, the processor, and the communication interface.

14. The sensing module of claim 13, wherein the conductive trace is on a tab configured to be manually torn to move the conductive trace from the first position to the second position.

15. The sensing module of claim 13, further comprising a switch operatively connected to the power source;
wherein with the conductive trace in the first position the switch is in an open position, and with the conductive trace in the second position the switch is in a closed position.

16. The sensing module of claim 1, wherein the sensor includes an accelerometer configured to gather data regarding vibration and spatial orientation.

17. The sensing module of claim 1, wherein the sensor includes a temperature sensor configured to gather data regarding temperature.

18. The sensing module of claim 1, wherein the sensor is configured to gather data regarding date, time, and at least one of vibration, temperature, sound, motion, humidity, pressure, fluid level, force, location, proximity, and spatial orientation.

19. The sensing module of claim 1, wherein the drug delivery device is either a drug delivery device containing a drug therein configured to be delivered from the drug delivery device or a drug delivery training device configured to simulate drug delivery therefrom.

20. A drug delivery system, comprising:
a drug delivery device; and
a sensing module configured to be attached to an outer surface of the drug delivery device, the sensing module including:
a sensor configured to gather data regarding at least one of date, time, vibration, temperature, sound, motion, humidity, pressure, fluid level, force, location, proximity, and spatial orientation;
a communication interface configured to wirelessly transmit data to an external source; and
a processor configured to receive data from the sensor indicative of the gathered data and to cause the communication interface to wirelessly transmit data indicative of the received data to the external source.

21. The system of claim 20, further comprising a flexible circuit board with the sensor, the processor, and the communication interface thereon.

22. The system of claim 20, further comprising a rigid circuit board with the sensor, the processor, and the communication interface thereon.

23. The system of claim 20, wherein the sensing module includes a housing with the sensor, the processor, and the communication interface disposed therein, the housing being attached to the outer surface of the drug delivery device.

24. The system of claim 23, wherein the sensing module includes a circuit board with the sensor, the processor, and the communication interface thereon, the circuit board being disposed within the housing.

25. The system of claim 20, wherein the sensing module includes a thin-film device, and the system further comprises an adhesive configured to attach the thin-film device to the outer surface of the drug delivery device.

26. The system of claim 20, wherein the sensing module is non-removably attached to the outer surface of the drug delivery device.

27. The system of claim 20, wherein the sensing module includes a power source configured to provide power to at least one of the sensor, the processor, and the communication interface.

28. The system of claim 27, further comprising an insulator in a first position, in which the insulator prevents the power source from providing the power to at least one of the sensor, the processor, and the communication interface;
wherein the insulator is configured to be manually moved by a user from the first position to a second position, in which the insulator allows the power source to provide the power to at least one of the sensor, the processor, and the communication interface.

29. The system of claim 28, wherein the drug delivery device includes a cap configured to be manually removed by a user from a housing of the drug delivery device, the removal of the cap being configured to cause the insulator to automatically move from the first position to the second position.

30. The system of claim 28, wherein the drug delivery device includes a trigger configured to be manually actuated by a user to trigger drug delivery from the drug delivery device, the actuation of the trigger being configured to cause the insulator to automatically move from the first position to the second position.

31. The system of claim 28, wherein the insulator includes a tab configured to be manually torn to move from the first position to the second position.

32. The system of claim 28, further comprising a switch operatively connected to the power source;
wherein with the insulator in the first position the switch is in an open position, and with the insulator in the second position the switch is in a closed position.

33. The system of claim 28, wherein the insulator includes a first tab;
the system further comprises a second tab in a third position, in which the sensor is not gathering the data;
the second tab is configured to be manually moved by a user from the third position to a fourth position; and
the movement of the second tab from the third position to the fourth position allows the sensor to begin gathering the data.

34. The system of claim 27, further comprising a conductive trace configured to be manually moved by a user from a first position, in which the conductive trace prevents the power source from providing the power to at least one of the sensor, the processor, and the communication interface to a second position, in which the conductive trace allows the power source to provide the power to at least one of the sensor, the processor, and the communication interface.

35. The system of claim 34, wherein the drug delivery device includes a cap configured to be manually removed by a user from a housing of the drug delivery device, the removal of the cap being configured to cause the conductive trace to automatically move from the first position to the second position.

36. The system of claim 34, wherein the drug delivery device includes a trigger configured to be manually actuated by a user to trigger drug delivery from the drug delivery device, the actuation of the trigger being configured to cause the conductive trace to automatically move from the first position to the second position.

37. The system of claim 34, wherein the conductive trace is on a tab configured to be manually torn to move the conductive trace from the first position to the second position.

38. The system of claim 34, further comprising a switch operatively connected to the power source;
wherein with the insulator in the first position the switch is in an open position, and with the insulator in the second position the switch is in a closed position.

39. The system of claim 27, wherein the drug delivery device includes a cap configured to be manually removed by a user from a housing of the drug delivery device, the removal of the cap being configured to cause the power source to begin providing power to the at least one of the sensor, the processor, and the communication interface.

40. The system of claim 20, wherein the sensor includes an accelerometer configured to gather data regarding vibration and spatial orientation.

41. The system of claim 20, wherein the sensor includes a temperature sensor configured to gather data regarding temperature.

42. The system of claim 20, wherein the sensor is configured to gather data regarding date, time, and at least one of vibration, temperature, sound, motion, humidity, pressure, fluid level, force, location, proximity, and spatial orientation.

43. The system of claim 20, wherein the drug delivery device is either a drug delivery device containing a drug therein configured to be delivered from the drug delivery device or a drug delivery training device configured to simulate drug delivery therefrom.

44. The system of claim 20, wherein the drug includes one of infliximab, golimumab, ustekinumab, daratumumab, guselkumab, epoetin alfa, risperidone, and paliperidone palmitate.

45-80. (canceled)
